(19) United States
(12) Patent Application Publication
(10) Pub. No.: US 2025/0353890 A1
(43) Pub. Date: Nov. 20, 2025

(54) CLEC9A-BASED CHIMERIC PROTEIN COMPLEXES

(71) Applicants: Orionis Biosciences, Inc., Waltham, MA (US); Orionis Biosciences BV, Zwijnaarde (BE)

(72) Inventors: Nikolai KLEY, Waltham, MA (US); Erik DEPLA, Zwijnaarde (BE); Lennart ZABEAU, Zwijnaarde (BE); Jan TAVERNIER, Zwijnaarde (BE)

(21) Appl. No.: 19/214,818

(22) Filed: May 21, 2025

Related U.S. Application Data
(63) Continuation of application No. 17/598,573, filed on Sep. 27, 2021, now Pat. No. 12,351,614.
(60) Provisional application No. 62/825,584, filed on Mar. 28, 2019.
(60) Provisional application No. 62/906,442, filed on Sep. 26, 2019.

(51) Int. Cl. C07K 14/56; A61K 38/00; A61P 35/00; C07K 14/00

(52) U.S. Cl. CPC C07K 14/56; A61P 35/00; C07K 14/001; A61K 38/00; C07K 2319/30

(57) ABSTRACT

The present invention relates, in part, to chimeric protein complexes including an anti-ClecSA targeting moiety, modified Fc domain, and a modified human IFNα and their use as therapeutic agents. The present invention further relates to pharmaceutical compositions comprising the chimeric protein complexes and their use in the treatment of various diseases.

### CROSS-REFERENCE TO RELATED APPLICATIONS

This application claims the benefit of and priority to U.S. Provisional Patent Application No. 62/906,442, filed Sep. 26, 2019, and to U.S. Provisional Patent Application No. 62/825,584, filed Mar. 28, 2019, the content of which are hereby incorporated by reference in their entirety.

### FIELD

The present invention relates, in part to, chimeric protein complexes that include a fragment crystallizable domain (Fc), a Clec9A VHH as a targeting moiety, and a modified interferon α2 (IFNα2) as a signaling agent. Use of these chimeric protein complexes as therapeutic agents is also disclosed.

### SEQUENCE LISTING

The instant application contains a Sequence Listing that has been submitted in ASCII format via EFS-Web and is hereby incorporated by reference in its entirety. Said ASCII copy, created on Mar. 26, 2020, is named ORN-063PC_ ST25.txt and is 139,264 bytes in size.

### BACKGROUND

Biologics with an effector function are a class of biologics that have many potential therapeutic applications. In some instances, these biologics, e.g., cytokines, encode an effector functions that can be systemically toxic if administered to humans. Accordingly, maximizing tolerability and therapeutic index of these biologics in humans is important so that systemic toxicity in humans or subjects can be reduced.

Often, these biologics need be delivered to their target(s) inside a subject with high precision and in a regulated manner in order for them to be effective. Thus, there is a need for engineering biological molecules that have high inherent safety profile, have the ability to reach their target inside the subject with high precision, and are able to function in a regulated fashion.

One example of such biologics, is a chimeric protein having a signaling agent (having an effector function, e.g., a cytokine), connected to a targeting element (having the ability to seek its target with high precision). In these biologics, the signaling agent can be a wild type signaling agent or a modified signaling agent (e.g. by mutation). The modified signaling agent is, generally, modified to cause an attenuation of the signaling agent's activity (e.g., substantially reducing its ability to interact with/engage its receptor) in a manner such that the signaling agent's effector function can be recovered upon binding of the targeting element to its target (e.g., antigen on target cell).

However, such chimeric proteins are amenable to therapeutic use only if certain conditions are met, e.g., the ability to be produced in a large scale, an in vivo half-life that ensures adequate time of exposure to the drug to elicit a therapeutically beneficial effect, a proper size to avoid rapid clearance or limited tissue penetrance and bio-distribution, and other properties that ensure adequate solubility, stability and storage without significant loss of function. Importantly all, or substantially most, of the above properties should be achieved without a loss of the conditional targeting of the effector function and retention of conditional engagement of a modified signaling agent with its receptor. Often, it is difficult to achieve all these objectives with chimeric proteins encoded or represented by a single, contiguous polypeptide chain. There is a need in the art where such desirable properties of the biologic can be achieved while maintaining the tolerability and therapeutic index of the biologic.

### SUMMARY

The present technology provides chimeric protein complexes that comprise biological therapeutic agents whose effector function can be delivered in a highly precise fashion to a target of choice, with limited or no cross-reactivities, and with limited of no systemic adverse events, while also providing features that impart pharmaceutical properties enabling the production of therapeutic agents with, for example, desired in vivo exposure time (e.g. half-life), size (e.g. for biodistribution and clearance characteristics), as well as large scale production and/or purification for commercial production (e.g. having adequate solubility, stability and storage properties).

In one aspect, the present invention relates to a heterodimeric protein complex and its individual polypeptide chain subunits (components), and where the protein complex includes a targeting moiety that specifically binds to C-type lectin domain family 9 member A (Clec9A), a modified human IFNα2, and a modified Fc domain.

In an aspect, the present invention is related to a chimeric protein complex comprising: (i) a targeting moiety that specifically binds to C-type lectin domain family 9 member A (Clec9A), (ii) a modified human IFNα2, and (iii) a modified Fc domain.

In one aspect, the present invention relates to a chimeric protein complex where the chimeric protein complex includes a targeting moiety that specifically binds to C-type lectin domain family 9 member A (Clec9A), a modified human IFNα2, and a modified Fc domain.

In some embodiments, the chimeric protein complex comprises a polypeptide having at least 95% identity with any one of SEQ ID NOs: 1-4 and 43 or at least 98% identity with any one of SEQ ID NOs: 1-4 and 43 or at least 99% identity with any one of SEQ ID Nos: 1-4 and 43. In some embodiments, the chimeric protein complex comprises a polypeptide of any one of SEQ ID NOs: 1-4 and 43, optionally with 0, or 1, or 2, or 3, or 4, or 5 mutations. In some embodiments, the chimeric protein complex comprises a polypeptide of any one of SEQ ID NOs: 1-4 and 43.

In some embodiments, the chimeric protein complex comprises a polypeptide incorporating a contiguous amino acid sequence having at least 95% identity with any one of SEQ ID NOs: 1-4 and 43 or at least 98% identity with any one of SEQ ID NOs: 1-4 and 43.

In another aspect, the present invention relates to a method of treating or preventing a cancer, comprising administering an effective amount of the chimeric protein complex, as disclosed herein, to a patient in need thereof.

Another aspect of the present invention relates to a pharmaceutical composition comprising a chimeric protein complex, as disclosed herein, and a pharmaceutically acceptable carrier. In another aspect, the present invention relates to a method for treating or preventing a cancer, comprising administering an effective amount of the pharmaceutical composition as disclosed herein to a patient in need thereof. In another aspect, the present invention relates to a recombinant nucleic acid composition encoding one or more of the polypeptide chain subunits of chimeric protein complexes disclosed herein. In another aspect, the present invention relates to a host cell including a nucleic acid composition encoding one or more chimeric protein complexes disclosed herein.

### BRIEF DESCRIPTION OF THE DRAWINGS

FIG. 1 shows various non-limiting illustrative schematics of the chimeric protein complexes of the present invention. In embodiments, each schematic is a composition of the present invention. Here “IFN” refers IFNα2, as described herein; “VHH” refers to anti-Clec9A VHH, as described herein; “ [symbol] ” is an optional “linker” as described herein; and the two long parallel rectangles with one having a protrusion and the other having an indentation are human Fc domains from IgG1, with knob-in-hole mutations as described herein and, optionally, with effector knock-out and/or stabilization mutations as also described herein. Although SEQ ID Nos are shown, these are illustrative only, e.g. they will change if alternative mutations besides R149A, as described herein, are used.

FIG. 2 shows plasma concentrations of Fc-AFNs after intravenous administration in mouse. Average values of 3 individual samples per time point time (+SEM) are plotted.

FIG. 3 shows the plasma concentrations of a CLEC9A AFN (construct lacking Fc) after intravenous administration in mouse. Average values of 3 individual samples per time point time (+SEM) are plotted.

FIG. 4A-D show specific binding of CLEC9A-AFN Fc-construct to cells expressing human CLEC9A (HL116-hCLEC9a) compared to control cells (HL116 and HEK293T).

FIG. 5 shows tumor growth curves in humanized mice after treatment with buffer or four different CLEC9A-AFN Fc-constructs. Average values (in mm³) of 5 animals per time point time (+SEM) are plotted.

FIG. 6 shows tumor growth curves in humanized mice after treatment with buffer or increasing doses of a single CLEC9A-AFN Fc-construct. Average values (in mm³) of 5 animals per time point time (+SEM) are plotted.

FIG. 7 shows various bivalent orientations and/or configurations that are encompassed by the present invention. The second VHH moiety to achieve bivalency is shaded and forms together with the attached linker a N- or C-terminal extension of the SEQ ID mentioned in the figure. Although SEQ ID Nos are shown, these are illustrative only, e.g. they will change if alternative mutations besides R149A, as described herein, are used. Further, the VHHs may be identical. See FIG. 1 description for clarification.

FIG. 8A shows results of pSTAT1 phosphorylation in Clec9A−/CD141− and Clec9A+/CD141+ PBMC's by IFNa2.

FIG. 8B shows results of pSTAT1 phosphorylation in Clec9A−/CD141− and Clec9A+/CD141+ PBMC's by AFNs with the A145G or M148A mutations.

FIG. 9 shows tumor growth curves in humanized mice after treatment with buffer or a 7.5 μg dose of two different CLEC9A-AFN Fc-constructs. Average values (+SEM) of tumor sizes (in mm³) of 6 animals per time point time were plotted.

FIGS. 10A-C show pSTAT1 activity in CLEC9A−/CD141− and CLEC9A+/CD141+ PBMC's after treatment with Clec9A targeted AFNs with (FIG. 10A) and without T106 O-glycosylation in IFNα2 (FIG. 10B), and an untargeted variant (FIG. 10C).

FIG. 11 shows anti-tumor activity of Clec9A targeted-AFN Fc with the A145G mutation of IFNα2.

### DETAILED DESCRIPTION OF THE INVENTION

In one aspect, the present invention relates to a chimeric protein complex where the chimeric protein complex includes a targeting moiety that specifically binds to C-type lectin domain family 9 member A (Clec9A), a modified human IFNα2, and a modified Fc domain. In embodiments, the chimeric protein complex comprises a polypeptide having at least 95% identity with any one of SEQ ID NOs: 1-4 and 43. In embodiments, the chimeric protein complex comprises a polypeptide having at least 98% identity with any one of SEQ ID NOs: 1-4 and 43 or at least 99% identity with any one of SEQ ID Nos: 1-4 and 43. In embodiments, the chimeric protein complex comprises a polypeptide of SEQ ID NOs: 1-4 and 43 wherein the sequence has less than 10 mutations as compared to the selected sequence. In embodiments, the chimeric protein complex comprises a polypeptide of SEQ ID NOs: 1-4 and 43 wherein the sequence has less than 5 mutations as compared to the selected sequence.

In embodiments, the chimeric protein complex comprises a polypeptide that has an amino acid sequence of SEQ ID NO: 1. This sequence includes a single domain antibody (VHH) against Clec9A (i.e., R1CHCL50(opt4), a linker (i.e., 5*GGS), and a Fc hole Ridgway sequence with LALA-KQ mutation (i.e., Fc hole Ridgway (LALA-KQ), see Ridgway et al., Protein Engineering 1996;9:617-621, which is incorporated by reference in its entirety). This construct of sequence of SEQ ID NO: 1 is denoted as follows: Variation 1 VHH-Fc R1CHCL50(opt4)-5*GGS-Fc hole Ridgway (LALA-KQ).

In embodiments, the chimeric protein complex comprises a polypeptide that has an amino acid sequence of SEQ ID NO: 2. This sequence includes a single domain antibody (VHH) against Clec9A (i.e., R1CHCL50(opt4), a linker (i.e., 5*GGS), and a Fc hole Merchant sequence with LALA-KQ mutation (i.e., Fc hole Merchant (LALA-KQ), see Merchant et al., Nature Biotechnology 1998;16:677-681, which is incorporated by reference in its entirety). This construct of SEQ ID NO: 2 is denoted as follows: VHH-Fc: R1CHCL50(opt4)-5*GGS-Fc hole Merchant (LALA-KQ).

In embodiments, the chimeric protein complex comprises a polypeptide that has an amino acid sequence of SEQ ID NO: 3. This sequence includes a single domain antibody (VHH) against Clec9A (i.e., 3LEC89(opt4)), a linker (i.e., 5*GGS), and a Fc hole Ridgway sequence with LALA-KQ mutation (i.e., Fc hole Ridgway (LALA-KQ), see Ridgway et al., Protein Engineering 1996;9:617-621, which is incorporated by reference in its entirety). This construct of SEQ ID NO: 3 is denoted as follows: VHH-Fc: 3LEC89(opt4)-5*GGS-Fc hole Ridgway (LALA-KQ)

In embodiments, the chimeric protein complex comprises a polypeptide that has an amino acid sequence of SEQ ID NO: 4. This sequence includes a single domain antibody (VHH) against Clec9A (i.e., 3LEC89(opt4), a linker (i.e., 5*GGS), and a Fc hole Merchant sequence with LALA-KQ mutation (i.e., Fc hole Merchant (LALA-KQ), see Merchant et al., Nature Biotechnology 1998; 16:677-681, which is incorporated by reference in its entirety). This construct of SEQ ID NO: 4 is denoted as follows: VHH-Fc: 3LEC89(opt4)-5*GGS-Fc hole Merchant (LALA-KQ).

In embodiments, the chimeric protein complex comprises a polypeptide that has an amino acid sequence of SEQ ID NO: 43. This sequence includes a single domain antibody (VHH) against Clec9A (i.e., R1CHCL50(opt4)), a linker (i.e., 5*GGS), and a Fc hole Merchant sequence with LALA-KQ mutation and without C terminal lysine (i.e., Fc hole Merchant (LALA-KQ), see Merchant et al., Nature Biotechnology 1998;16:677-681, which is incorporated by reference in its entirety).

The chimeric protein complex of the present invention may further include an amino acid sequence having at least 95% identity with any one of SEQ ID NOs: 5-8, 29-36, or 41-42. In embodiments, the chimeric protein complex comprises a polypeptide that has an amino acid sequence of at least 98% identity with any one of SEQ ID NOs: 5-8, 29-36, or 41-42or at least 99% identity with any one of SEQ ID Nos: 5-8, 29-36, or 41-42. In embodiments, the chimeric protein complex comprises a polypeptide that has an amino acid sequence selected from SEQ ID NOs: 5-8, 29-36, or 41-42wherein the sequence has less than 10 mutations as compared to the selected sequence. In embodiments, the chimeric protein complex comprises a polypeptide that has an amino acid sequence selected from SEQ ID NOs: 5-8, 29-36, or 41-42 wherein the sequence has less than 5 mutations as compared to the selected sequence.

In embodiments, the chimeric protein complex comprises a polypeptide that has an amino acid sequence of SEQ ID NO: 5. This sequence includes a modified human interferon a2b having a R149A mutations (i.e., huIFNa2B_R149A), a linker (i.e., 10*GGS-G), and a Fc knob Ridgway sequence with LALA-KQ mutation (i.e., Fc knob Ridgway (LALA-KQ), see Ridgway et al., Protein Engineering 1996;9:617-621, which is incorporated by reference in its entirety). This construct of sequence of SEQ ID NO: 5 is denoted as follows: Variation 1 Fc-AFN: Fc knob Ridgway (LALA-KQ)-10*GGS-G-huIFNa2B_R149A.

In embodiments, the chimeric protein complex comprises a polypeptide that has an amino acid sequence of SEQ ID NO: 6. This sequence includes a modified human interferon α2b having R149A and T106E mutations (i.e., huIFNa2B_R149A_T106E), a linker (i.e., 10*GGS-G), and a Fc knob Ridgway sequence with LALA-KQ mutation (i.e., Fc knob Ridgway (LALA-KQ), see Ridgway et al., Protein Engineering 1996;9:617-621, which is incorporated by reference in its entirety). This construct of sequence of SEQ ID NO: 6 is denoted as follows: Variation 2 Fc-AFN: Fc knob Ridgway (LALA-KQ)-10*GGS-G-huIFNa2B_R149A_T106E.

In embodiments, the chimeric protein complex comprises a polypeptide that has an amino acid sequence of SEQ ID NO: 7. This sequence includes a modified human interferon α2b having a R149A mutations (i.e., huIFNa2B_R149A), a linker (i.e., 10*GGS-G), and a Fc knob Merchant sequence with LALA-KQ mutation (i.e., Fc knob Merchant (LALA-KQ), see Merchant et al., Nature Biotechnology 1998;16:677-681, which is incorporated by reference in its entirety). This construct of sequence of SEQ ID NO: 7 is denoted as follows: Variation 3 Fc-AFN: Fc knob Merchant (LALA-KQ)-10*GGS-G-huIFNa2B_R149A.

In embodiments, the chimeric protein complex comprises a polypeptide that has an amino acid sequence of SEQ ID NO: 8. This sequence includes a modified human interferon α2b having R149A and T106E mutations (i.e., huIFNa2B_R149A_T106E), a linker (i.e., 10*GGS-G), and a Fc knob Merchant sequence with LALA-KQ mutation (i.e., Fc knob Merchant (LALA-KQ), see Merchant et al., Nature Biotechnology 1998;16:677-681, which is incorporated by reference in its entirety). This construct of sequence of SEQ ID NO: 8 is denoted as follows: Variation 4 Fc-AFN: Fc knob Merchant (LALA-KQ)-10*GGS-G-huIFNa2B_R149A_T106E.

In embodiments, the chimeric protein complex comprises a polypeptide that has an amino acid sequence of SEQ ID NO: 41. This sequence includes a modified interferon α2b having A145G mutation, a linker (i.e., 10*GGS-G), and a Fc knob Merchant sequence with LALA-KQ mutation (i.e., Fc knob Merchant (LALA-KQ), see Merchant et al., Nature Biotechnology 1998; 16:677-681, which is incorporated by reference in its entirety). This construct of sequence of SEQ ID NO: 41 is denoted as follows: Fc4′-IFNa2b_A145G.

In embodiments, the chimeric protein complex comprises a polypeptide that has an amino acid sequence of SEQ ID NO: 42. This sequence includes a modified interferon α2b having T106A and A145G mutations, a linker (i.e., 10*GGS-G), and a Fc knob Merchant sequence with LALA-KQ mutation (i.e., Fc knob Merchant (LALA-KQ), see Merchant et al., Nature Biotechnology 1998;16:677-681, which is incorporated by reference in its entirety). This construct of sequence of SEQ ID NO: 42 is denoted as follows: Fc4′-IFNa2a_T106E_A145G.

In one embodiment, the chimeric protein complex includes (i) an amino acid sequence having at least 95% identity with any one of SEQ ID NOs: 1 or 3 and (ii) an amino acid sequence having at least 95% identity with any one of SEQ ID NOs: 5 or 6. In another embodiment, the chimeric protein complex includes (i) an amino acid sequence having at least 98% identity with any one of SEQ ID NOs: 1 or 3 and (ii) an amino acid sequence having at least 98% identity with any one of SEQ ID NOs: 5 or 6. In another embodiment, the chimeric protein complex includes (i) an amino acid sequence having at least 99% identity with any one of SEQ ID NOs: 1 or 3 and (ii) an amino acid sequence having at least 99% identity with any one of SEQ ID NOs: 5 or 6. In an embodiment, the chimeric protein complex includes (i) an amino acid sequence having at least 95% identity with any one of SEQ ID NOs: 2 or 4 and (ii) an amino acid sequence having at least 95% identity with any one of SEQ ID NOs: 7 or 8. In an embodiment, the chimeric protein complex includes (i) an amino acid sequence having at least 98% identity with any one of SEQ ID NOs: 2 or 4 and (ii) an amino acid sequence having at least 98% identity with any one of SEQ ID NOs: 7 or 8. In another embodiment, the chimeric protein complex includes (i) an amino acid sequence having at least 99% identity with any one of SEQ ID NOs: 2 or 4 and (ii) an amino acid sequence having at least 99% identity with any one of SEQ ID NOs: 7 or 8.

In some embodiments, the chimeric protein complex comprises (i) a polypeptide having an amino acid sequence having at least 95% identity with SEQ ID NO: 2 and (ii) a polypeptide having an amino acid sequence having at least 95% identity with any one of SEQ ID NOs: 31 or 32. In some embodiments, the chimeric protein complex comprises (i) a polypeptide having an amino acid sequence having at least 98% identity with SEQ ID NO: 2 and (ii) a polypeptide having an amino acid sequence having at least 98% identity with any one of SEQ ID NOs: 31 or 32. In embodiments, the chimeric protein complex comprises (i) a polypeptide having an amino acid sequence having at least 99% identity with SEQ ID NO: 2 and (ii) a polypeptide having an amino acid sequence having at least 99% identity with any one of SEQ ID NOs: 31 or 32. In some embodiments, the chimeric protein complex comprises (i) a polypeptide having an amino acid sequence having at least 95% identity with SEQ ID NO: 43 and (ii) a polypeptide having an amino acid sequence having at least 95% identity with any one of SEQ ID NOs: 41 or 42. In some embodiments, the chimeric protein complex comprises (i) a polypeptide having an amino acid sequence having at least 98% identity with SEQ ID NO: 43 and (ii) a polypeptide having an amino acid sequence having at least 98% identity with any one of SEQ ID NOs: 41 or 42. In embodiments, the chimeric protein complex comprises (i) a polypeptide having an amino acid sequence having at least 99% identity with SEQ ID NO: 43 and (ii) a polypeptide having an amino acid sequence having at least 99% identity with any one of SEQ ID NOs: 41 or 42.

In some embodiments, the chimeric protein complex comprises: (i) a polypeptide having an amino acid sequence having at least 95% identity with any one of SEQ ID NOs: 1 or 3 and (ii) a polypeptide having an amino acid sequence having at least 95% identity with any one of SEQ ID NOs: 5 or 6.

In some embodiments, the chimeric protein complex comprises: (i) a polypeptide having an amino acid sequence having at least 98% identity with any one of SEQ ID NOs: 1 or 3 and (ii) a polypeptide having an amino acid sequence having at least 98% identity with any one of SEQ ID NOs: 5 or 6.

In some embodiments, the chimeric protein complex comprises: (i) a polypeptide having an amino acid sequence having at least 99% identity with any one of SEQ ID NOs: 1 or 3 and (ii) a polypeptide having an amino acid sequence having at least 99% identity with any one of SEQ ID NOs: 5 or 6.

In some embodiments, the chimeric protein complex comprises: (i) a polypeptide having an amino acid sequence having at least 95% identity with any one of SEQ ID NOs: 2 or 4 and (ii) a polypeptide having an amino acid sequence having at least 95% identity with any one of SEQ ID NOs: 7 or 8.

In embodiments, the chimeric protein complex comprises: (i) a polypeptide having an amino acid sequence having at least 98% identity with any one of SEQ ID NOs: 2 or 4 and (ii) a polypeptide having an amino acid sequence having at least 98% identity with any one of SEQ ID NOs: 7 or 8.

In embodiments, the chimeric protein complex comprises: (i) a polypeptide having an amino acid sequence having at least 99% identity with any one of SEQ ID NOs: 2 or 4 and (ii) a polypeptide having an amino acid sequence having at least 99% identity with any one of SEQ ID NOs: 7 or 8.

In embodiments, the chimeric protein complex comprises: (i) a polypeptide having an amino acid sequence having at least 95% identity with SEQ ID NO: 2 and (ii) a polypeptide having an amino acid sequence having at least 95% identity with any one of SEQ ID NOs: 31 or 32.

In embodiments, the chimeric protein complex comprises: (i) a polypeptide having an amino acid sequence having at least 98% identity with SEQ ID NO: 2 and (ii) a polypeptide having an amino acid sequence having at least 98% identity with any one of SEQ ID NOs: 31 or 32.

In embodiments, the chimeric protein complex comprises: (i) a polypeptide having an amino acid sequence having at least 99% identity with SEQ ID NO: 2 and (ii) a polypeptide having an amino acid sequence having at least 99% identity with any one of SEQ ID NOs: 31 or 32.

In embodiments, the chimeric protein complex comprises: (i) a polypeptide having an amino acid sequence having at least 95% identity with SEQ ID NO: 43 and (ii) a polypeptide having an amino acid sequence having at least 95% identity with any one of SEQ ID NOs: 41 or 42.

In embodiments, the chimeric protein complex comprises: (i) a polypeptide having an amino acid sequence having at least 98% identity with SEQ ID NO: 43 and (ii) a polypeptide having an amino acid sequence having at least 98% identity with any one of SEQ ID NOs: 41 or 42.

In some embodiments, the chimeric protein complex comprises: (i) a polypeptide having an amino acid sequence having at least 99% identity with SEQ ID NO: 43 and (ii) a polypeptide having an amino acid sequence having at least 99% identity with any one of SEQ ID NOs: 41 or 42.

In some embodiments, the present invention is related to a multivalent or bivalent chimeric protein complex comprising: a polypeptide having a sequence at least 95%, or 97%, or 98%, or 99% identical to the amino acid sequence of SEQ ID NO: 17 and a polypeptide having a sequence at least 95%, or 97%, or 98%, or 99% identical to the amino acid sequence of SEQ ID NO: 7; a polypeptide having a sequence at least 95%, or 97%, or 98%, or 99% identical to the amino acid sequence of SEQ ID NO: 2 and a polypeptide having a sequence at least 95%, or 97%, or 98%, or 99% identical to the amino acid sequence of SEQ ID NO: 19; a polypeptide having a sequence at least 95%, or 97%, or 98%, or 99% identical to the amino acid sequence of SEQ ID NO: 18 and a polypeptide having a sequence at least 95%, or 97%, or 98%, or 99% identical to the amino acid sequence of SEQ ID NO: 7; a polypeptide having a sequence at least 95%, or 97%, or 98%, or 99% identical to the amino acid sequence of SEQ ID NO: 20 and a polypeptide having a sequence at least 95%, or 97%, or 98%, or 99% identical to the amino acid sequence of SEQ ID NO: 7; a polypeptide having a sequence at least 95%, or 97%, or 98%, or 99% identical to the amino acid sequence of SEQ ID NO: 2 and a polypeptide having a sequence at least 95%, or 97%, or 98%, or 99% identical to the amino acid sequence of SEQ ID NO: 22; or a polypeptide having a sequence at least 95%, or 97%, or 98%, or 99% identical to the amino acid sequence of SEQ ID NO: 21 and a polypeptide having a sequence at least 95%, or 97%, or 98%, or 99% identical to the amino acid sequence of SEQ ID NO: 7. In some embodiments, the present invention is related to a method for treating or preventing a cancer, comprising administering an effective amount of the multivalent or bivalent chimeric protein complex described herein to a patient in need thereof.

In some embodiments, the present invention is related to a chimeric protein complex comprising at least two polypeptides having a sequence at least 95%, or 97%, or 98%, or 99%, or 100% identical to the amino acid sequence of any one of SEQ ID Nos: 1-43. In embodiments, the present invention is related to a method for treating or preventing a cancer, comprising administering an effective amount of the chimeric protein complex comprising at least two polypeptides having a sequence at least 95%, or 97%, or 98%, or 99%, or 100% identical to the amino acid sequence of any one of SEQ ID Nos: 1-43 to a patient in need thereof.

In some embodiments, the chimeric protein complex includes a modified human interferon α2. In embodiments, the modified IFN-α2 agent has reduced affinity and/or activity for the IFN-α/β receptor (IFNAR), i.e., IFNAR1 and/or IFNAR2 chains. In some embodiments, the modified IFN-α2 agent has substantially reduced or ablated affinity and/or activity for the IFN-α/β receptor (IFNAR), i.e., IFNAR1 and/or IFNAR2 chains. In some embodiments, the modified human interferon α2, as disclosed herein, has an amino acid sequence having at least 95% identity with of SEQ ID NOs: 9 or 10. In other embodiments, the modified human IFNα2 has an amino acid sequence having at least 98% identity or at least 99% identity with of SEQ ID NOs: 9 or 10. In some embodiments, the modified human IFNα2 has 1-3 mutations relative to the amino acid sequence of SEQ ID NOs: 9 or 10. In one embodiment, the modified human IFNα2 comprises a R149A mutation with respect to SEQ ID NOs: 9 or 10. In one embodiment, the modified human IFNα2 comprises a A145G mutation with respect to SEQ ID NOs: 9 or 10.

In some embodiments, the targeting moiety of the chimeric protein complex disclosed herein comprises a recombinant heavy-chain-only antibody (VHH). In some embodiments, the VHH has an amino acid sequence of at least 95% identity with of one of SEQ ID NOs: 11 or 12. In other embodiments, the VHH has an amino acid sequence of at least 98% identity with of one of SEQ ID NOs: 11 or 12 or at least 99% identity with of one of SEQ ID NOs: 11 or 12. In some embodiments, the VHH has an amino acid sequence of any one of SEQ ID NOs: 11 and 12.

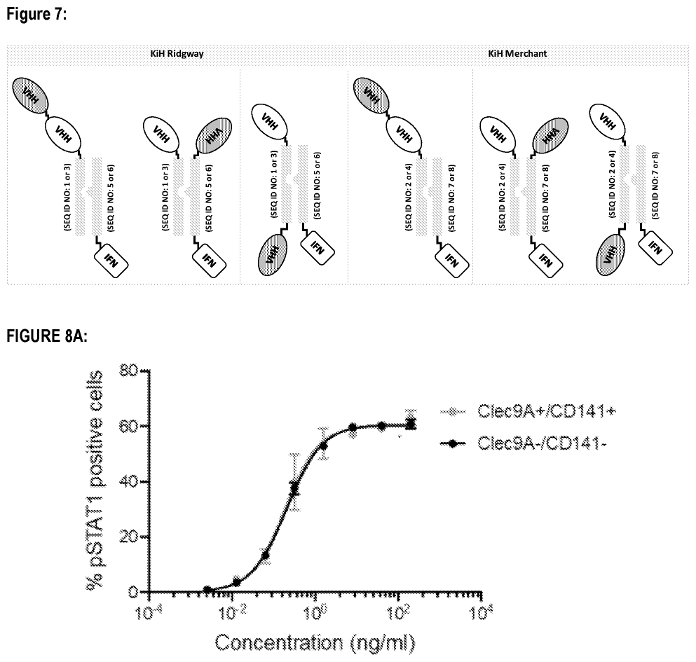

In some embodiments, the chimeric protein complex disclosed herein comprises two targeting moieties. In some embodiments, the chimeric protein complex disclosed herein comprises two identical targeting moieties. In embodiments, these bivalent modes are oriented as shown in FIG. 7.

In some embodiments, the chimeric protein complex disclosed herein comprises two targeting moieties. In some embodiments, the chimeric protein complex disclosed herein comprises two non-identical targeting moieties. In embodiments, these bivalent modes are oriented as shown in FIG. 7. For example, in some embodiments, the chimeric protein complex disclosed herein comprises targeting moieties (without limitation, VHHs) against Clec9A and PD-L1.

In embodiments, the R149A mutation is present in the IFN-α2.

In embodiments, the R149A mutation is not present in the IFN-α2 and instead, another mutation is present. For instance, this alternative mutation could be at one of positions R33, R144, A145, M148, and L153. In embodiments, the alternative mutation is one of R33A, R144A, R1441, R144L, R144S, R144T, R144Y, A145D, A145G, A145H, A145K, A145Y, M148A, and L153A. For clarity, in embodiments, any reference to R149A herein may be replaced with one of R33A, R144A, R1441, R144L, R144S, R144T, R144Y, A145D, A145G, A145H, A145K, A145Y, M148A and L153A In embodiments, any reference to R149A herein may be replaced with A145G.

In some embodiments, the chimeric protein complex disclosed herein include at least one Fc domain. In some embodiments, the chimeric protein complex includes a modified Fc domain where the modified Fc domain includes one or more of the following mutations: P329G, K322Q, K322A, or P331S relative to any of one of SEQ ID NO: 13-16. In other embodiments, the modified Fc domain includes one or more of the following mutations: P329G, K322Q, K322A, or P331S relative to human IgG1 Fc.

In some embodiments, the chimeric protein complex includes a modified Fc domain that has an amino acid sequence having at least 90% identity with SEQ ID NO: 13-16. In other embodiments, the modified Fc domain has an amino acid sequence having at least 93% identity with SEQ ID NO: 13-16. In other embodiments, the modified Fc domain has an amino acid sequence having at least 95% identity with SEQ ID NO: 13-16.

In another aspect, the present invention relates to a method of treating or preventing a cancer, comprising administering an effective amount of the chimeric protein complex, as disclosed herein, to a patient in need thereof. The method can be used to treat or prevent cancers selected from one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

Another aspect of the present invention relates to a pharmaceutical composition comprising a chimeric protein complex, as disclosed herein, and a pharmaceutically acceptable carrier. In some embodiments, the present invention pertains to pharmaceutical compositions comprising the present chimeric protein complex.

Another aspect of the present invention relates to a method for treating or preventing a cancer, comprising administering an effective amount of the pharmaceutical composition as disclosed herein to a patient in need thereof. The pharmaceutical composition can be used for the treatment or prevention of a cancer selected from one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

In another aspect, the present invention relates to a recombinant nucleic acid composition encoding one or more chimeric protein complexes disclosed herein, e.g. encoding the entire chimeric protein complex or constituent polypeptides thereof. In another aspect, the present invention relates to a host cell including the recombinant nucleic acid composition complexes disclosed herein.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

As used herein, the term “effective amount” refers to a quantity sufficient to achieve a desired therapeutic and/or prophylactic effect, e.g., an amount which results in the prevention of, or a decrease in a disease or disorder or one or more signs or symptoms associated with a disease or disorder. In the context of therapeutic or prophylactic applications, the amount of a composition administered to the subject will depend on the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder. As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

As used herein, Fc domain mutations are numbered according to EU convention (Edelman et al., PNAS 1969; 63 (1) 78-85, incorporated by reference in its entirety). As used herein, the term “LALA” mutation refers to a double mutant Fc domain having L234A mutation and a L235A mutation. As used herein, the term “KQ” mutation refers to a mutant Fc domain having a K322Q mutation.

Knob in hole mutants are those described in Ridgway et al., Protein Engineering 1996;9:617-621, which is hereby incorporated by reference in its entirety, i.e. Y407T/T366Y.

Alternatively, knob in hole mutants are those described in Merchant et al., Nature Biotechnology 1998;16:677-681,which is incorporated by reference in its entirety, i.e. S354C: T366W/Y349C: T366S: L368A: Y407V.

Unless noted, the Fc is from human IgG1.

SEQUENCES

SEQ ID NO: 1 Variation 1 VHH-Fc R1CHCL50(opt4)-5*GGS-Fc hole Ridgway (LALA-KQ)
DVQLVESGGGLVQPGGSLRLSCAASGSFSSINVMGWYRQAPGKERELVARITNLGLPNYADSVKGRFTISRDNSKNTV
YLQMNSLRPEDTAVYYCYLVALKAEYWGQGTLVTVSSGGSGGSGGSGGSGGSDKTHTCPPCPAPEAAGGPSVFLFPPK
PKDTLMISRTPEVTCVVVDVSHEDPEVKFNWYVDGVEVHNAKTKPREEQYNSTYRVVSVLTVLHQDWLNGKEYKCQVS
NKALPAPIEKTISKAKGQPREPQVYTLPPSRDELTKNQVSLTCLVKGFYPSDIAVEWESNGQPENNYKTTPPVLDSDG
SFFLTSKLTVDKSRWQQGNVFSCSVMHEALHNHYTQKSLSLSPGK
SEQ ID NO: 2 Variation 2 VHH-Fc: R1CHCL50(opt4)-5*GGS-Fc hole Merchant
(LALA-KQ)
DVQLVESGGGLVQPGGSLRLSCAASGSFSSINVMGWYRQAPGKERELVARITNLGLPNYADSVKGRFTISRDNSKNTV
YLQMNSLRPEDTAVYYCYLVALKAEYWGQGTLVTVSSGGSGGSGGSGGSGGSDKTHTCPPCPAPEAAGGPSVFLFPPK
PKDTLMISRTPEVTCVVVDVSHEDPEVKFNWYVDGVEVHNAKTKPREEQYNSTYRVVSVLTVLHQDWLNGKEYKCQVS
NKALPAPIEKTISKAKGQPREPQVCTLPPSRDELTKNQVSLSCAVKGFYPSDIAVEWESNGQPENNYKTTPPVLDSDG
SFFLVSKLTVDKSRWQQGNVFSCSVMHEALHNHYTQKSLSLSPGK
SEQ ID NO: 3 Variation 3 VHH-Fc: 3LEC89(opt4)-5*GGS-Fc hole Ridgway (LALA-KQ)
DVQLVESGGGLVQPGGSLRLSCAASGRIFSVNAMGWYRQAPGKQRELVAAITNQGAPTYADSVKGRFTISRDNSKNTV
YLQMNSLRPEDTAVYYCKAFTRGDDYWGQGTLVTVSSGGSGGSGGSGGSGGSDKTHTCPPCPAPEAAGGPSVFLFPPK
PKDTLMISRTPEVTCVVVDVSHEDPEVKFNWYVDGVEVHNAKTKPREEQYNSTYRVVSVLTVLHQDWLNGKEYKCQVS
NKALPAPIEKTISKAKGQPREPQVYTLPPSRDELTKNQVSLTCLVKGFYPSDIAVEWESNGQPENNYKTTPPVLDSDG
SFFLTSKLTVDKSRWQQGNVFSCSVMHEALHNHYTQKSLSLSPGK
SEQ ID NO: 4 Variation 3 VHH-Fc: 3LEC89(opt4)-5*GGS-Fc hole Ridgway (LALA-KQ)
DVQLVESGGGLVQPGGSLRLSCAASGRIFSVNAMGWYRQAPGKQRELVAAITNQGAPTYADSVKGRFTISRDNSKNTV
YLQMNSLRPEDTAVYYCKAFTRGDDYWGQGTLVTVSSGGSGGSGGSGGSGGSDKTHTCPPCPAPEAAGGPSVFLFPPK
PKDTLMISRTPEVTCVVVDVSHEDPEVKFNWYVDGVEVHNAKTKPREEQYNSTYRVVSVLTVLHQDWLNGKEYKCQVS
NKALPAPIEKTISKAKGQPREPQVCTLPPSRDELTKNQVSLSCAVKGFYPSDIAVEWESNGQPENNYKTTPPVLDSDG
SFFLVSKLTVDKSRWQQGNVFSCSVMHELAHNHYTQKSLSLSPGK
SEQ ID NO: 5 Variation 1 Fc-AFN: Fc knob Ridgway (LALA-KQ)-10*GGS-G-
huIFNa2B_R149A
DKTHTCPPCPAPEAAGGPSVFLFPPKPKDTLMISRTPEVTCVVVDVSHEDPEVKFNWYVDGVEVHNAKTKPREEQYNS
TYRVVSVLTVLHQDWLNGKEYKCQVSNKALPAPIEKTISKAKGQPREPQVYTLPPSRDELTKNQVSLYCLVKGFYPSD
IAVEWESNGQPENNYKTTPPVLDSDGSFFLYSKLTVDKSRWQQGNVFSCSVMHEALHNHYTQKSLSLSPGKGGSGGSG
GSGGSGGSGGSGGSGGSGGSGGSGCDLPQTHSLGSRRTLMLLAQMRRISLFSCLKDRHDFGFPQEEFGNQFQKAETIP
VLHEMIQQIFNLFSTKDSSAAWDETLLDKFYTELYQQLNDLEACVIQGVGVTETPLMKEDSILAVRKYFQRITLYLKE
KKYSPCAWEVVRAEIMASFSLSTNLQESLRSKE
SEQ ID NO: 6 Variation 2 Fc-AFN: Fc knob Ridgway (LALA-KQ)-10*GGS-G-
huIFNa2B_R149A_T106E
DKTHTCPPCPAPEAAGGSPVFLFPPKPKDTLMISRTEPVTCVVVDVSHEDPEVKFNWYVDGVEVHNAKTKPREEQYNS
TYRVVSVLTVLHQDWLNGKEYKCQVSNKALPAPIEKTISKAKGQPREPQVYTLPPSRDELTKNQVSLYCLVKGFYPSD
IAVEWESNGQPENNYKTTPPVLDSDGSFFLYSKLTVDKSRWQQGNVFSCSVMHEALHNHYTQKSLSLSPGKGGSGGSG
GSGGSGGSGGSGGSGGSGGSGGSGCDLPQTHSLGSRRTLMLLAQMRRISLFSCLKDRHDFGFPQEEFGNQFQKAETIP
VLHEMIQQIFNLFSTKDSSAAWDETLLDKFYTELYQQLNDLEACVIQGVGVEETPLMKEDSILAVRKYFQRITLYLKE
KKYSPCASEVVRAEIMASFSLSTNLQESLRSKE
SEQ ID NO: 7 Variation 3 Fc-AFN: Fc knob Merchant (LALA-KQ)-10*GGS-G-
huIFNa2B_R149A
DKTHTCPPCPAPEAAGGPSVFLFPPKPKDTLMISRTPEVTCVVVDVSHEDPEVKFNWYVDGVEVHNAKTKPREEQYNS
TYRVVSVLTVLHQDWLNGKEYKCQVSNKALPAPIEKTISKAKGQPREPQVYTLPPCRDELTKNQVSLWCLVKGFYPSD
IAVEWESNGQPENNKYTTPPVLDSDGSFFLYSKLTVDKSRWQQGNVFSCSVMHEALHNHYTQKSLSLSPGKGGSGGSG
GSGGSGGSGGSGGSGGSGGSGGSGCDLPQTHSLGSRRTLMLLAQMRRISLFSCLKDRHDFGFPQEEFGNQFQKAETIP
VLHEMIQQIFNLFSTKDSSAAWDETLLDKFYTELYQQLNDLEACVIQGVGVTETPLMKEDSILAVRKYFQRITLYLKE
KKYSPCASEVVRAEIMASFSLSTNLQESLRSKE
SEQ ID NO: 8 Variation 4 Fc-AFN: Fc knob Merchant (LALA-KQ)-10*GGS-G-
huIFNa2B_R149A_T106E
DKTHTCPPCPAPEAAGGPSVFLFPPKPKDTLMISRTPEVTCVVVDVSHEDPEVKFNWYVDGVEVHNAKTKPREEQYNS
TYRVVSVLTVLHQDWLNGKEYKCQVSNKALPAPIEKTISKAKGQPREPQVYTLPPCRDELTKNQVSLWCLVKGFYPSD
IAVEWESNGQPENNYKTTPPVLDSDGSFFLYSKLTVDKSRWQQGNVFSCSVMHEALHNHYTQKSLSLSPGKGGSGGSG
GSGGSGGSGGSGGSGGSGGSGGSGCDLPQTHSLGSRRTLMLLAQMRRISLFSCLKDRHDFGFPQEEFGNQFQKAETIP
VLHEMIQQIFNLFSTKDSSAAWDETLLDKFYTELYQQLNDLEACVIQGVGVEETPLMKEDSILAVRKYFQRITLYLKE
KKYSPCAWEVVRAEIMASFSLSTNLQESLRSKE
SEQ ID NO: 9 Human IFNα2a (amino acid sequence)
CDLPQTHSLGSRRTLMLLAWMRKISLFSCLKDRHDFGFPQEEFGNQFQKAETIPVLHEMIQQIFNLFSTKDSSAAWDE
TLLDKFYTELYQQLNDLEACVIQGVGVTETPLMKEDSILAVRKYFQRITLYLKEKKYSPCAWEVVRAEIMRSFSLSTN
LQESLRSKE
SEQ ID NO: 10 Human IFNα2B (amino acid sequence)
CDLPQTHSLGSRRTLMLLAQMRRISLFSCLKDRHDFGFPQEEFGNQFQKAETIPVLHEMIQQIFNLFSTKDSSAAWDE
TLLDKFYTELYQQLNDLEACVIQGVGVTETPLMKEDSILAVRKYFQRITLYLKEKKYSPCAWEWRAEIMRSFSLSTNL
QESLRSKE
SEQ ID NO: 11 R1CHCL50_opt4 (anti-human Clec9a VHH)
DVQLVESGGGLVQPGGSLRLSCAASGSFSSINVMGWYRQAPGKERELVARITNLGLPNYADSVKGRFTISRDNSKNTV
YLQMNSLRPEDTAVYYCYLVALKAEYWGQGTLVTVSS
SEQ ID NO: 12 3LEC89_opt4 (anti-human Clec9a VHH)
DVQLVESGGGLVQPGGSLRLSCAASGRIFSVNAMGWYRQAPGKKQRELVAAITNQGAPTYADSVKGRFTISRDNSKNT
VYLQMNSLRPEDTAVYYCKAFTRGDDYWGQGTLVTVSS
SEQ ID NO: 13 Amino acid sequence of the Fc (human IgG1)-with LALA mutations
and Ridgway hole
DKTHTCPPCPAPEAAGGPSVFLFPPKPKDTLMISRTPEVTCVVVDVSHEDPEVKFNWYVDGVEVHNAKTKPREEQYNS
TRYVVSVLTVLHQDWLNGKEYKCKVSNKALPAPIEKTISKAKGQPREPQVYTLPPSRDELTKNQVSLTCLVKGFYPSD
IAVEWESNGQPENNYKTTPPVLDSDGSFFLTSKLTVKDSRWQQGNVFSCSVMHEALHNHYTQKSLSLSPGK
SEQ ID NO: 14 Amino acid sequence of the Fc (human IgG1)-with LALA mutations
and Ridgway knob
DKTHTCPPCPAPEAAGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTKNQVSLYCLVKGFYPSD
IAVEWESNGQPENNYKTTPPVLDSDGSFFLYSKLTVDKSRWQQGNVFSCSVMHEALHNHYTQKSLSLSPGK
SEQ ID NO: 15 Amino acid sequence of the Fc (human IgG1)-with LALA mutations
and Merchant hole
DKTHTCPPCPAPEAAGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVCTLPPSRDELTKNQVSLSCAVKGFYPSD
IAVEWESNGQPENNYKTTPPVLDSDGSFFLVSKLTVDKSRWQQGNVFSCSVMHEALHNHYTQKSLSLSPGK
SEQ ID NO: 16 Amino acid sequence of the Fc (human IgG1)-with LALA mutations
and Merchant knob
DKTHTCPPCPAPEAAGGPSVFLFPPKPKDTLMISRPTEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPCRDELTKNQVSLWCLVKGFYPSD
IAVEWESNGQPENNYKTTPPVLDSDGSFFLYSKLTVDKSRWQQGNVFSCSVMHEALHNHYTQKSLSLSPGK

Other sequences are identified elsewhere in the text.

### EXAMPLES

In some Examples, two variants of the knob-in-hole technology are used: Ridgway (derived from Ridgway et al., Protein Engineering 1996;9:617-621) and Merchant (derived from Merchant et al., Nature Biotechnology 1998;16:677-681). The ‘standard’ effector-mutation in the Ridgway constructs is LALA-PG (P329G) and this is noted herein. The ‘standard’ effector-mutation in the Merchant constructs is LALA-KQ (K322Q) and this is noted herein.

The terms “ActaFeron (AFN),” or “ActaKine” are occasionally used herein to reference a chimeric protein described herein (details are provided in the Examples regarding the format of the chimeric protein).

### Example 1: Fc-based AcTaferons

In order to increase the half-life of CLEC9A specific (CLEC9A is a highly specific cDC1 marker) AcTaferons human CLEC9A-VHH_huIFNa2 fusion proteins were converted into an Fc-fusion. For this purpose, the human IgG1-Fc was fused via a 20*GGS linker to the AcTaferon (VHH 3LEC89-20*GGS-huIFNa2_R149). In a second version the Fc domain was constructed in between the VHH and the IFN moiety. Effector functions of the human IgG1-Fc are reduced by introducing the LALA-P329G mutation.

The relevant sequences for expression in mammalian cells are:

P-956: pcDNA3.4-mouse light chain kappa-hIgG1-LALA-PG-20*GGS-3LEC89-20*GGS+G-IFNa2_R149A
(SEQ ID NO: 23)
MKLPVRLLVLMFWIPASSSDKTHTCPPCPAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALGAPIEKTISKAKGQPREPQVYTLPP
SRDELTKNQVSLTCLVKGFYPSDIAVEWESNGQPENNYKTTPPVLDSDGSFFLYSKLTVDKSRWQQGNVFSCSVM
HEALHNHYTQKSLSLSPGKGGSGGSGGSGGSGGSGGSGGSGGSGGSGGSGGSGGSGGSGGSGGSGGSGGSG
GSGGSGGSQVQLQESGGGLVQPGGSLRLSCAASGRIFSVNAMGWYRQAPGKQRELVAAITNQGAPTYADSVKGRF
TISRDNAGNTVYLQMNSLRPEDTAVYYCKAFTRGDDYWGQGTQVTVSSGGSGGSGGSGGSGGSGGSGGSGGSG
GSGGSGGSGGSGGSGGSGGSGGSGGSGGSGGSGGSGCDLPQTHSLGSRRTLMLLAQMRKISLFSCLKDRHDFG
FPQEEFGNQFQKAETIPVLHEMIQQIFNLFSTKDSSAAWDETLLDKFYTELYQQLNDLEACVIQGVGVTETPLMKEDSI
LAVRKYFQRITLYLKEKKYSPCAWEVVRAEIMASFSLSTNLQESLRSKE.
P-957: pcDNA3.4-mouse Ig heavy chain-3LEC89-20*GGS-hIgG1-LALA-PG-20*GGS+G-IFNa2_R149A
(SEQ ID NO: 24)
MGWSCIIFFLVATATGVHSQVQLQESGGGLVQPGGSLRLSCAASGRIFSVNAMGWYRQAPGKQRELVAAITNQGAP
TYADSVKGRFTISRDNAGNTVYLQMNSLRPEDTAVYYCKAFTRGDDYWGQGTQVTVSSGGSGGSGGSGGSGGSG
GSGGSGGSGGSGGSGGSGGSGGSGGSGGSGGSGGSGGSGGSGGSDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQDWLNGKEYKCKV
SNKALGAPIEKTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGKGGSGGSGGSGGSGGSGGSGGSGGSG
GSGGSGGSGGSGGSGGSGGSGGSGGSGGSGGSGGSGCDLPQTHSLGSRRTLMLLAQMRKISLESCLKDRHDFG
FPQEEFGNQFQKAETIPVLHEMIQQIFNLFSTKDSSAAWDETLLDKFYTELYQQLNDLEACVIQGVGVTETPLMKEDSI
LAVRKYFQRITLYLKEKKYSPCAWEWVRAEIMASESLSTNLQESLRSKE.

The constructs were made by GeneArt (Thermo Fisher) and transiently expressed in the ExpiCHO expression system (Thermo Fisher) according to the manufacturer's guidelines. Ten days after transfection, supernatant was collected and cells removed by centrifugation. Recombinant proteins were purified from the medium using the rProtein A Sepharose Fast Flow resin (GE Healthcare) according to the manufacturer's guidelines. Unexpectedly the proteins although expressed at 70-170 mg/L showed severe solubility problems and even at concentrations below 1 mg/ml they tended to aggregate and precipitate when stored at 4° C. or after a single freeze-thaw cycle. When the VHH 3LEC89 was replaced by the unrelated VHH 2LIG99 specific for human PD-L1 similar observations were made indicating that an Fc-based AcTakine format has manufacturability liabilities.

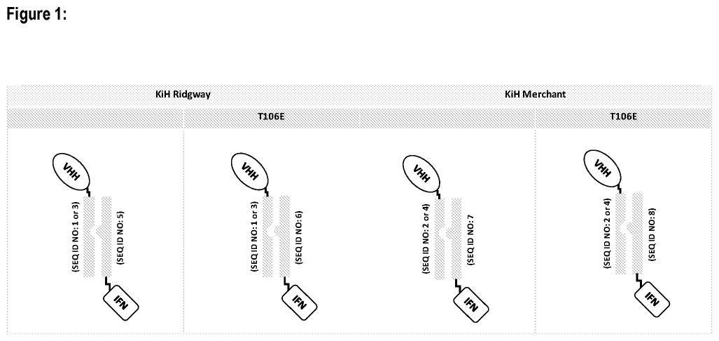

Surprisingly the solubility problem was solved by designing a different type of Fc-construct. In this new format a heterodimeric Fc complex is generated by combining a VHH-Fc fusion with a Fc-IFN fusion using either the knob into hole mutations Y407T/T366Y or S354C: T366W/Y349C: T366S: L368A: Y407V. Additional variants include an optional knock-out of the O-glycosylation site in the huIFNa2 by the T106E mutation. In total 8 constructs were designed based on 2 different humanized human CLEC9A specific VHH. To reduce effector functions of the IgG1-Fc protein the mutation LALA-K322Q was used. The sequences of the mature proteins are represented by SEQ ID NO: 1-8. This result in total in 8 different Fc-complexes that can be generated by combining knob and hole constructs as shown in FIG. 1.

For expression in mammalian cells the sequences are linked to a leader sequence and constructs were made by GeneArt (Thermo Fisher). Production was performed in ExpiCHO cells as described above. Recombinant proteins were purified from the supernatant on a HiTrap Protein A HP (GE Healthcare) and eluted proteins were, after neutralization, desalted on a G25 column (GE Healthcare) followed by final and 0.22 μm filtration. Proteins showed to remain soluble at 4° C. or after repeated freeze/thaw cycles at concentrations of at least 10 mg/mL.

### Example 2: PK Effects of Chimera with or without Fc

PK (pharmacokinetics) study in mouse with the 4 different variants of the R1CHCL50 based Fc-proteins.

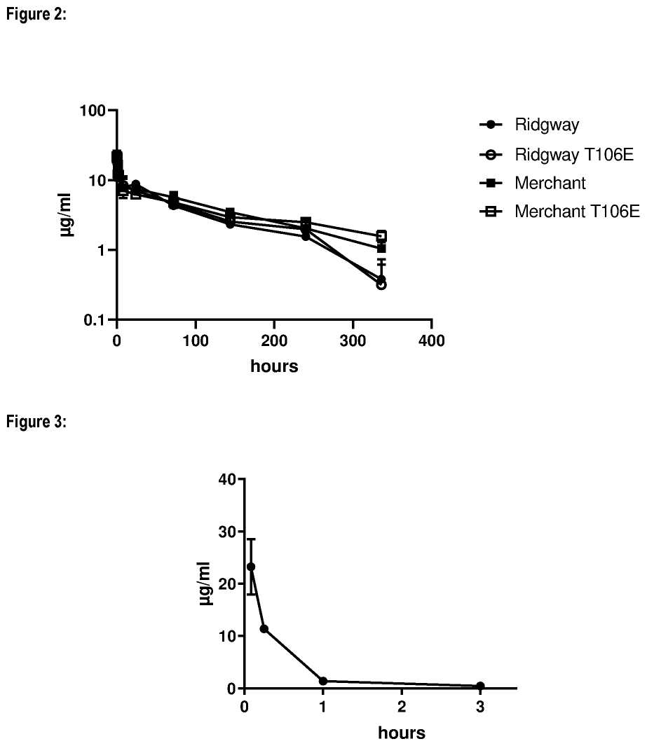

In total 9 mice were dosed intravenously at 1 mg/kg with each construct. K-EDTA blood was taken from a first group of 3 mice at 5 minutes, 8 hours and 6 days, from a second group of 3 mice at 15 minutes, 1 day and 10 days and from a third group of 3 mice at 2 hours, 3 days and 14 days. The concentration of intact CLEC9A-AFN Fc-construct was measured by ELISA. In brief the MAXISORP Nunc Immune plates (Thermo Scientific) were coated overnight with anti-human interferon alpha mAb (clone MMHA-13; PBL Assay Science) at 0.5 μg/ml in PBS. After washing the plates four times with PBS +0,05% Tween-20, they were blocked with 0.1% Casein in PBS for at least 1 hour at room temperature. Subsequently, diluted samples and standards were incubated in 0.1% Casein in PBS for 2 hours at room temperature. After another wash cycle a custom made rabbit-anti-VHH (diluted 1/20000 in 0.1% Casein in PBS) was incubated for 2 hours at room temperature followed by an additional wash cycle and incubation with HRP-conjugated goat anti-rabbit (Jackson—111-035-144; 1:5000 in 0.1% Casein) for 1 hour at room temperature. After a final washing cycle, peroxidase activity was measured using KPL substrate (5120-0047; SeraCare) according to the manufacturer's instructions. Concentrations from samples were calculated using GraphPad Prism. Measured concentrations are plotted in FIG. 2 and show that all 4 constructs have a similar PK profile except for a somewhat faster clearance of the Ridgway based Fc-construct at the last sampling time point. Terminal half-life was estimated on average at about 3 days for the Ridgway constructs and 4.5 days for the Merchant constructs.

PK study in mouse with a CLEC9A AcTaferon without Fc-fusion.

In a separate study the PK of an AFN without Fc (3LEC89-20*GGS-huIFNa2_R149A-his6) in mice was evaluated. This chimera has the sequence of:

P-602 sequence
(SEQ ID NO: 25)
QVQLQESGGGLVQPGGSLRLSCAASGRIFSVNAMGWYRQAPGKQRELVAA
ITNQGAPTYADSVKGRFTISRDNAGNTVYLQMNSLRPEDTGAVYYCKAFT
RGDDYWGQGTQVTVSSVDGGSGGSGGSGGSGGSGGSRSGGSGGSGGSGGS
GGSGGSGGSGGSGGSGGSGGSGGSGGSAAAMCDLPQTHSLGSRRTLMLLA
QMRRISLFSCLKDRDHFGFPQEEFGNQFQKAETIPVLHEMIQQIFNLFST
KDSSAAWDETLLDKFYTELYQQLNDLEACVIQGVGVTETPLMKEDSILAV
RKYFQRITLYLKEKKYSPCAWEVVRAEIMASFSLSTNLQESLRSKELEHH
HHHH.

Nine animals were dosed intravenously at 3 mg/kg. K-EDTA blood was taken from a first group of 3 mice at 5 minutes and 1 hour, from a second group of 3 mice at 15 minutes and 3 hours and finally from the last group at 8 hours. The concentration in the plasma samples was measured using the same ELISA as described for the Fc-fusion proteins. The measured concentration (FIG. 3) show a fast clearing of this type of molecules resulting in a concentration below detection limit (0.12 μg/ml) at the 8-hour time point. The estimated terminal half-life is in the range of only 2 hours, clearly demonstrating the superior half-life properties of the Fc-based AcTakines.

### Example 3: Binding and In Vivo Effects of Constructs

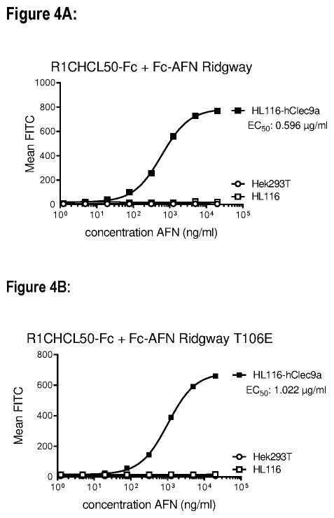

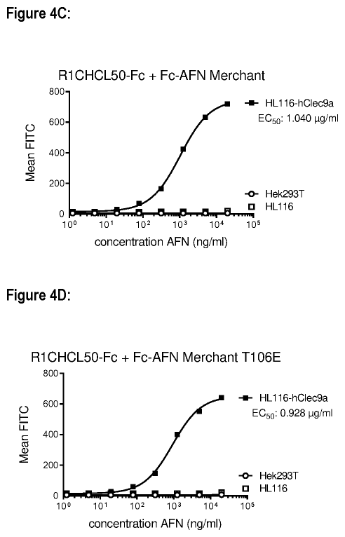

To measure relative binding affinities the same 4 molecules as shown in Example 2 were incubated with a serial dilution of CLEC9A-AFN Fc-construct on HL116-hClec9A cells. To asses binding specificity also parental HL116 cells and parental HEK293T cells (both lacking detectable expression of Clec9A) were incubated with an identical serial dilution of the CLEC9A-AFN Fc-construct. Binding was detected by subsequent incubation with an FITC-coupled anti-human secondary Ab, measured on a MACSQuant X instrument (Miltenyi Biotech) and analyzed using the FlowLogic software (Miltenyi Biotech). Data in FIG. 4 illustrates that the Fc-based AFNs have similar binding EC50s for HL116-hClec9A cells while no binding was detected on the cell lines not expressing Clec9A.

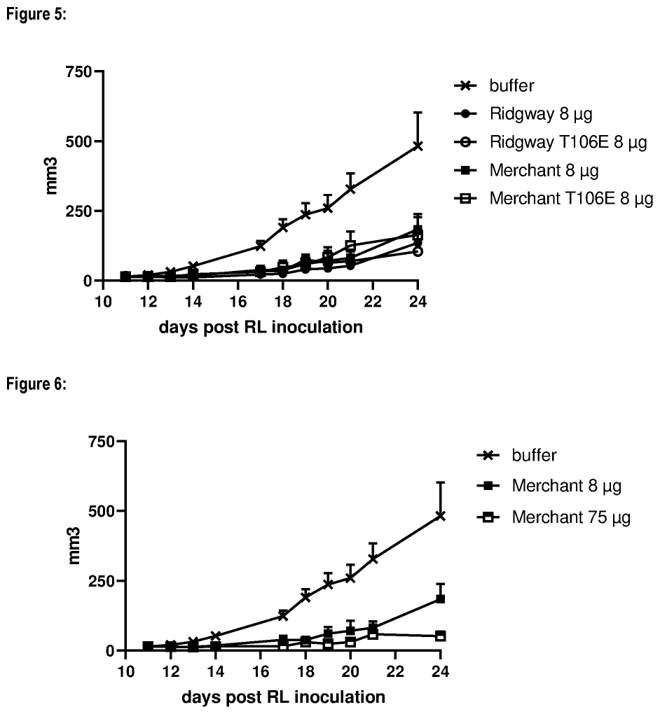

To evaluate the efficacy of the Fc-based AFNs the molecules were tested in a tumor model in a humanized mouse. In brief, newborn NSG mice (1-2 days of age) were sublethal irradiated with 100 cGy prior to intrahepatic delivery of 1×10⁵ CD34+ human stem cells (from HLA-A2 positive cord bloods). At week 13 after stem cell transfer mice were subcutaneously inoculated with 25×10⁵ human RL follicular lymphoma cells (ATCC CRL-2261; not sensitive to the direct anti-proliferative effect of IFN). Mice were treated daily intraperitoneally with 30 μg of human Flt3L protein, from day 10 to day 19 after tumor inoculation. Weekly intravenous injection with buffer or Fc-AFN (8 or 75 μg) was initiated at day 11 after tumor inoculation, when a palpable tumor was visible (n=5 mice per group). Tumor size (caliper measurements), body weight and temperature were assessed daily. Data in FIGS. 5 and FIG. 6 show the tumor growth until 6 days after the second treatment. FIG. 5 demonstrates that all constructs induced a similar level of tumor growth inhibition at the lower dose of 8 μg. FIG. 6 shows the result of higher doses for a Merchant construct resulting in increasing tumor growth inhibition. Data on body weight and temperature did not show any major difference between buffer treatment and AFN treatment supporting that all AFN treatments were well tolerated.

### Example 4: Bivalent and Bispecific Variants

In order to further increase the targeting capacity of the molecules, additional VHH moieties are added resulting in constructs of which non-limiting examples of configurations are shown in FIG. 7. These novel constructs target CLEC9A in a bivalent mode or co-target e.g. both CLEC9A and PD-L1. By way of example constructs below are based on the R1CHCL50 (opt4) VHH directed against CLEC9A and/or the 2LIG99 VHH directed against PD-L1 and the huIFNa2B_R149A. By replacing R1CHCL50 (opt4) with 3LEC89 (opt4) a similar series can be generated. Alternatively the huIFNa2B_R149A can be replaced with huIFNa2B_R149A_T106E. Finally examples below are based on Fc moieties containing Merchant based knob-into-hole mutations which can be exchanged for Fc moieties with Ridgway based knob-into-hole mutations.

Novel constructs
A. Hole chain for bivalent CLEC9A targeting by
additional VHH at N-terminus of Fc (Merchant)
VHH-VHH-Fc: R1CHCL50(opt4)-5*GGS-R1CHCL50(opt4)-
5*GGS-Fc (LALA-KQ)
(SEQ ID NO: 17)
DVQLVESGGGLVQPGGSLRLSCAASGSFSSINVMGWYRQAPGKERELVAR
ITNLGLPNYADSVKGRFTISRDNSKNTVYLQMNSLRPEDTAVYYVYLVAL
KAEYWGQGTLVTVSSGGSGGSGGSGGSGGSDVQLVESGGGLVQPGGSLRL
SCAASGSFSSINVMGWYRQAPGKERELVARITNLGLPNYADSVKGRFTIS
RDNSKNTVYLQMNSLRPEDTAVYYCYLVALKAEYWGQGTLVTVSSGGSGG
SGGSGGSGGSDKTHTCPPCPAPEAAGGPSVFLFPPKPKDTLMISRTPEVT
CVVVDVSHEDPEVKFNWYVDGVEVHNAKTKPREEQYNSTYRVVSVLTVLH
QDWLNGKEYKCQVSNKALPAPIEKTISKAKGQPREPQVCTLPPSRDELTK
NQVSLSCAVKGFYPSDIAVEWESNGQPENNYKTTPPVLDSDGSFFLVSKL
TVDKSRWQQGNVFSCSVHEALHNHYTQKSLSLSPGK
B. Hole chain for bivalent CLEC9A targeting by
additional VHH on C-terminus of Fc (Merchant)
VHH-Fc-VHH: R1CHCL50(opt4)-5*GGS-Fc (LALA-KQ)-
5*GGS-R1CHCL50(opt4)
(SEQ ID NO: 18)
DVQLVESGGGLVQPGGSLRLSCAASGSFSSINVMGWYRQAPGKERELVAR
ITNLGLPNYADSVKGRFTISRDNSKNTVYLQMNSLRPEDTAVYYCYLVAL
KAEYWGQGTLVTVSSGGSGGSGGSGGSGGSDKTHTCPPCPAPEAAGGPSV
FLFPPKPKDTLMISRTPEVTCVVVDVSHEDPEVKFNWYVDGVEVHNAKTK
PREEQYNSTYRVVSVLTVLHQDWLNGKEYKCQVSNKALPAPIEKTISKAK
GQPREPQVCTLPPSRDELTKNQVSLSCAVKGFYPSDIAVEWESNGQPENN
YKTTPPVLDSDGSFFLVSKLTVDKSRWQQGNVFSCSVMHEALHNHYTQKS
LSLSPGKGGSGGSGGSGGSGGSDVQLVESGGGLVQPGGSLRLSCAASGSF
SSINVMGWYRQAPGKERELVARITNLGLPNYADSVKGRFTISRDNSKNTV
YLQMNSLRPEDTAVYYCYLVALKAEYWGQGTLVTVSS
C. Knob chain for bivalent CLEC9A targeting by
additional VHH on N-terminus of Fc (Merchant)
VHH-Fc-AFN: R1CHCL50(opt4)-5*GGS-Fc(LALA-KQ)-
10*GGS-G-huIFNa2B_R149A
(SEQ ID NO: 19)
DVQLVESGGGLVQPGGSLRLSCAASGSFSSINVMGWYRQAPGKERELVAR
ITNLGLPNYADSVKGRFTISRDNSKNTVYLQMNSLRPEDTAVYYCYLVAL
KAEYWGQGTLVTVSSGGSGGSGGSGGSGGSDKTHTCPPCPAPEAAGGPSV
FLFPPKPKDTLMISRTPEVTCVVVDVSHEDPEVKFNWYVDGVEVHNAKTK
PREEQYNSTYRVVSVLTVLHQDWLNGKEYKCQVSNKALPAPIEKTISKAK
GQPREPQVYTLPPCRDELTKNQVSLWCLVKGFYPSDIAVEWESNGQPENN
YKTTPPVLDSDGSFFLYSKLTVDKSRWQQGNVFSCSVMHEALHNHYTQKS
LSLSPGKGGSGGSGGSGGSGGSGGSGGSGGSGGSGCDLPQTHSLGSRRTL
MLLAQMRRISLFSCLKDRHDFGFPQEEFGNQFQKAETIPVLHEMIQQIFN
LFSTKDSSAAWDETLLDKFYTELQQLNDLEACVIQGVGVTETPLMKEDSI
LAVRKYFQRITLYLEKKYSPCAWEVVRAEIMASFSLSTNLQESLRSKE
D. Hole chain for bispecific CLEC9A-PDL1
targeting by additional VHH at N-terminus of Fc
(Merchant)
VHH-VHH-Fc: 2LIG99-5*GGS-R1CHCL50(opt4)-5*GGS-Fc
(LALA-KQ)
(SEQ ID NO: 20)
QVQLQESGGGLVQAGGSLRLSCTASGTIFSINRMDWFRQAPGKQRELVAL
ITSDGTPAYADSAKGRFTISRDNTKKTVSLQMNSLKPEDTAVYYCHVSSG
VYNYWGQGTQVTVSSGGSGGSGGSGGSGGSDVQLVESGGGLVQPGGSLRL
SCAASGSFSSINVMGWYRQAPGKERELVARITNLGLPNYADSVKGRFTIS
RDNSKNTVYLQMNSLRPEDTAVYYCYLVALKAEYWGQGTLVTVSSGGSGG
SGGSGGSGGSDKTHTCPPCPAPEAAGGPSVFLFPPKPKDTLMISRTPEVT
CVVVDVSHEDPEVKFNWYVDGVEVHNAKTKPREEQYNSTYRVVSVLTVLH
QDWLNGKEYKCQVSNKALPAPIEKTISKAKGQPREPQVCTLPPSRDELTK
NQVSLSCAVKGFYPSDIAVEWESNGQPENNYKTTPPVLDSGSFFLVSKLT
VDKSRWQQGNVFSCSVMHEALHNHYTQKSLSLSPGK
E. Hole chain for bispecific CLEC9A-PD-L1
targeting by additional VHH on C-terminus of Fc
(Merchant)
VHH-Fc-VHH: R1CHCL5-(opt4)-5*GGS-Fc (LALA-KQ)-
5*GGS-2LIG99
(SEQ ID NO: 21)
DVQLVESGGGLVQPGGSLRLSCAASGSFSSINVMGWYRQAPGKERELVAR
ITNLGLPNYADSVKGRFTISRDNSKNTVYLQMNSLRPEDTAVYYCYLVAL
KAEYWGQGTLVTVSSGGSGGSGGSGGSGGSDKTHTCPPCPAPEAAGGPSV
FLFPPKPKDTLMISRTPEVTCVVVDVSHEDPEVKFNWYVDGVEVHNAKTK
PREEQYNSTYRVVSVLTVLHQDWLNGKEYKCQVSNKALPAPIEKTISKAK
GQPREPQVCTLPPSRDELTKNQVSLSCAVKGFYPSIDAVEWESNGQPENN
YKTTPPVLDSDGSFFLVSKLTVDKSRWQQGNVFSCSVMHEALHNHYTQKS
LSLSPGKGGSGGSGGSGGSGGSQVQLQESGGGLVQAGGSLRLSCTASGTI
FSINRMDWFRQAPGKQRELVALITSDGTPAYADSAKGRFTISRDNTKKTV
SLQMNSLKPEDTAVYYCYVSSGVYNYWGQGTQVTVSS
F. Knob chain for bispecific CLEC9A-PD-L1
targeting by additional VHH on N-terminus of Fc
(Merchant)
VHH-Fc-AFN: 2LIG99-5*GGS-Fc(LALA-KQ)-10*GGS-G-
huIFNa2B_R149A
(SEQ ID NO: 22)
QVQLQESGGGLVQAGGSLRLSCTASGTIFSINRMDWFRQAPGKQRELVAL
ITSDGTPAYADSAKGRFTISRDNTKKTVSLQMNSLKPEDTAVYYCHVSSG
VYNYWGQGTQVTVSSGGSGGSGGSGGSGGSDKTHTCPPCPAPEAAGGPSV
FLFPPKPKDTLMISRTPEVTCVVVDVSHEDPEVKFNWYVDGVEVHNAKTK
PREEQYNSTYRVVSVLTVLHQDWLNGKEYKCQVSNKALPAPIEKTISKAK
GQPREPQVYTLPPCRDELTKNQVSLWCLVKGFYPSDIAVEWESNGQPENN
YKTTPPVLDSDGSFFLYSKLTVDKSRWQQGNVFSCSCMHEALHNHYTQKS
LSLSPGKGGSGGSGGSGGSGGSGGSGGSGGSGGSGGSGCDLPQTHSLGSR
RTLMLLAQMRRISLFSCLKDRHDFGFPQEEFGNQFQKAETIPVLHEMIQQ
IFNLFSTKDSSAAWDETLLDKFYTELYQQLNDLEACVIQGVGVTETPLMK
EDSILAVRKYFQRITLYLKEKKYSPCASEVVRAEIMASFSLSTNLQESLR
SKE

For expression in mammalian cells the sequences are linked to a leader sequence and expression constructs were made by GeneArt (Thermo Fisher). Production is performed in ExpiCHO cells as described above. Recombinant proteins are purified from the supernatant on a HiTrap Protein A HP (GE Healthcare) and eluted proteins are, after neutralization, desalted on a G25 column (GE Healthcare) followed by final and 0.22 μm filtration. More specifically the following expressions constructs are combined to generate the Fc-based AcTaferons
Construct containing SEQ ID NO: 17+construct containing SEQ ID NO: 7
Construct containing SEQ ID NO: 2+construct containing SEQ ID NO: 19
Construct containing SEQ ID NO: 18+construct containing SEQ ID NO: 7
Construct containing SEQ ID NO: 20+construct containing SEQ ID NO: 7
Construct containing SEQ ID NO: 2+construct containing SEQ ID NO: 22
Construct containing SEQ ID NO: 21+construct containing SEQ ID NO: 7

### Example 5: A145G and M148A AFN Mutations

In this example, the potential of the IFN variations A145G and M148A as AFN mutation (i.e. the warhead mutation that results in a loss in biological activity, which can be restored upon targeting of the warhead) was evaluated.

Mutations were evaluated in the heterodimeric, ‘knob-in-hole’ Fc AFN context. Here, the Clec9A VHH R1CHCL50 sequence was, via the flexible 20*GGS-linker and in the pcDNA3.4 expression vector, fused to the human IgG1 Fc sequence containing the L234A_L235A_K322Q effector mutations ‘hole’ modifications Y349C_T366S_L368A_Y407V (see sequence R1CHCL50-Fc3 below). Second AFN partner, also cloned in the pcDNA3.4 vector, consisted of the fusion between the human IgG1 Fc sequence containing the L234A_L235A_K322Q effector mutations and the ‘knob’ modifications S354C_T366W and the hIFNα2 sequence with the AFN mutation A145G or M148A and the O-glycosylation mutation T106E (see sequences below).

To produce these ‘knob-in-hole’ Fc AFNs, a combination of both ‘hole’ and ‘knob’ plasmids was transfected in ExpiCHO™ cells (ThermoFisher) according to the manufacturer's instructions. Seven days post transfection, recombinant proteins were purified using protein A spin plates (ThermoFisher), quantified and purity tested using SDS-PAGE.

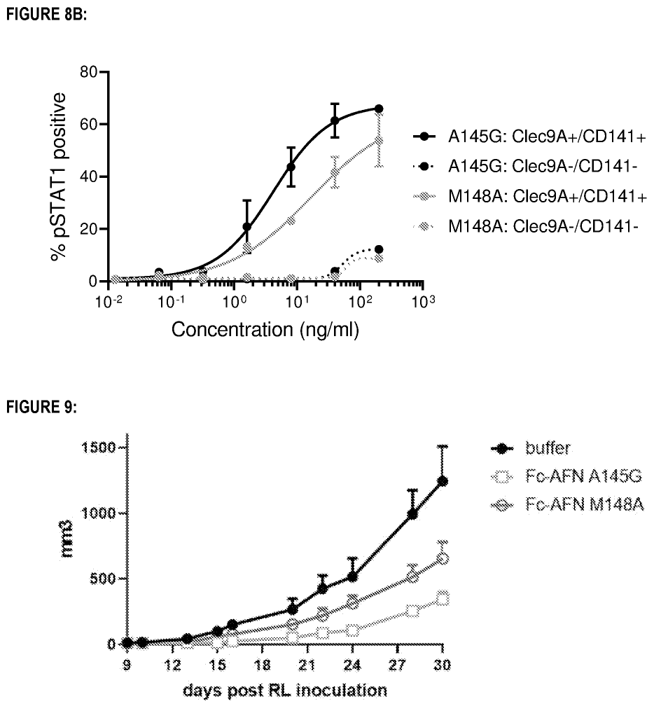

Resulting A145G and M148 AFN's were tested for STAT1 phosphorylation in primary cDC1 cells (expressing Clec9A, the target of the AFN's) compared to other PBMC populations. In brief, PBMCs from buffy coats of healthy donors were isolated using density gradient centrifugation using Lymphoprep™ (StemCell technologies). Cells were washed twice with FACS buffer (2% FBS, 1 mM EDTA in PBS) and stained with anti-Clec9A and anti-CD141 Abs (both Miltenyi) to identify the cDC1 population for 20 minutes at 4° C. After two washes, cells were stimulated with a serial dilution wild type IFNα2 or both AFN's for 15 minutes at 37° C. After fixation (10 minutes, 37° C., Fix Buffer I; BD Biosciences), permeabilization (30 minutes, on ice, Perm III Buffer I; BD Biosciences) and washing, cells were stained with anti-STAT1 pY701 Ab (BD Biosciences). Samples were acquired with a MACSQuant® X instrument (Miltenyi Biotec) and analyzed using the FlowLogic™ software (Miltenyi Biotec). Data in FIG. 8A-B clearly illustrate that (i) Clec9A−/CD141− and Clec9A+/CD141+ cells are comparable sensitive to wild type IFNα2, and that (ii) both A145G and M148A mutations abolishes most of the signaling in non-cDC1 PBMC's (Clec9A−/CD141−) but that targeting to Clec9A positive cells (Clec9A+/CD141+) to a great extent restores this signaling. This results in an AFN effect of at least 100-fold for both the A145G or M148A mutations and illustrates the potential of these mutations for the design of AFNs.

Sequences:
P-1451: R1CHCL50-20*GGS-hIgG1 Fc_L234A_L235A_K322Q_Y349C_T366S_L368A_Y407V
(short: R1CHCL50-Fc3)
(SEQ ID NO: 26)
QVQLQESGGGLVHPGGSLRLSCAASGSFSSINVMGWYRQAPGKERELVARITNLGLPNYADSVTGRETISRDN
AKNTVYLQMNSLKPEDTAVYYCYLVALKAEYWGQGTQVTVSSGGSGGSGGSGGSGGSGGSGGSGGSGGSG
GSGGSGGSGGSGGSGGSGGSGGSGGSGGSGGSDKTHTCPPCPAPEAAGGPSVFLFPPKPKDTLMISRTPEV
TCVVVDVSHEDPEVKFNWYVDGVEVHNAKTKPREEQYNSTYRVVSVLTVLHQDWLNGKEYKCQVSNKALPAPI
EKTISKAKGQPREPQVCTLPPSRDELTKNQVSLSCAVKGFYPSDIAVEWESNGQPENNYKTTPPVLDSDGSFFL
VSKLTVDKSRWQQGNVFSCSVMHEALHNHYTQKSLSLSPGK
P-1846: hIgG1 Fc_L234A_L235A_K322Q_S354C_T366W-20*GGS-hIFNa2_T106E_A145G
(short: Fc4-hIFNa2_A145G)
(SEQ ID NO: 27)
DKTHTCPPCPAPEAAGGPSVFLFPPKPKDTLMISRTPEVTCVVVDVSHEDPEVKFNWYVDGVEVHNAKTKPREE
QYNSTYRVVSVLTVLHQDWLNGKEYKCQVSNKALPAPIEKTISKAKGQPREPQVYTLPPCRDELTKNQVSLWCL
VKGFYPSDIAVEWESNGQPENNYKTTPPVLDSDGSFFLYSKLTVDKSRWQQGNVFSCSVMHEALHNHYTQKSL
SLSPGKGGSGGSGGSGGSGGSGGSGGSGGSGGSGGSGGSGGSGGSGGSGGSGGSGGSGGSGGSGGSC
DLPQTHSLGSRRTLMLLAQMRKISLFSCLKDRHDFGFPQEEFGNQFQKAETIPVLHEMIQQIFNLFSTKDSSAAW
DETLLDKFYTELYQQLNDLEACVIQGVGVEETPLMKEDSILAVRKYFORITLYLKEKKYSPCAWEVVRGEIMRSES
LSTNLQESLRSKE
P-1850: hIgG1 Fc_L234A_L235A_K322Q_S354C_T366W-20*GGS-hIFNa2_T106E_M148A
(short: Fc4-hIFNa2_M148A)
(SEQ ID NO: 28)
DKTHTCPPCPAPEAAGGPSVFLFPPKPKDTLMISRTPEVTCVVVDVSHEDPEVKFNWYVDGVEVHNAKTKPREE
QYNSTYRVVSVLTVLHQDWLNGKEYKCQVSNKALPAPIEKTISKAKGQPREPQVYTLPPCRDELTKNQVSLWCL
VKGFYPSDIAVEWESNGQPENNYKTTPPVLDSDGSFFLYSKLTVDKSRWQQGNVFSCSVMHEALHNHYTQKSL
SLSPGKGGSGGSGGSGGSGGSGGSGGSGGSGGSGGSGGSGGSGGSGGSGGSGGSGGSGGSGGSGGSC
DLPQTHSLGSRRTLMLLAQMRKISLFSCLKDRHDFGFPQEEFGNQFQKAETIPVLHEMIQQIFNLFSTKDSSAAW
DETLLDKFYTELYQQLNDLEACVIQGVGVEETPLMKEDSILAVRKYFQRITLYLKEKKYSPCAWEVVRAEIARSFS
LSTNLQESLRSKE

### Example 6: A145G and M148A AFN Mutations In Vivo

To evaluate the efficacy of the Fc-based AFNs the molecules were tested in a tumor model in a humanized mouse. In brief, new-born NSG mice (1-2 days of age) were sublethally irradiated with 100 cGy prior to intrahepatic delivery of 1×10⁵ CD34+ human stem cells (from HLA-A2 positive cord bloods). At week 13 after stem cell transfer mice were subcutaneously inoculated with 25×10⁵ human RL follicular lymphoma cells (ATCC CRL-2261; not sensitive to the direct anti-proliferative effect of IFN). Mice were treated intraperitoneally with 30 μg of human Flt3L protein, from day 9 to day 22 after tumor inoculation. Weekly intravenous injection with buffer or Fc-AFN (7.5 μg) constructs as described in example 5 was initiated at day 9 after tumor inoculation, when a palpable tumor was visible (n=6 mice per group). Tumor size (caliper measurements), body weight and temperature were assessed daily. Data in FIG. 9 show the tumor growth until one week after the third treatment and demonstrate that both constructs induced a strong level of tumor growth inhibition. Data on body weight and temperature did not show any major difference between buffer treatment and AFN treatment supporting that all AFN treatments were well tolerated.

### Example 7: Additional Fc-AFN Constructs based on A145G and M148A Mutations

The following additional Fc constructs with attenuated human interferon alpha2 were generated:

A. hIgG1 Fc_L234A_L235A_K322Q_S354C_T366W-10*GGS-G -hIFNa2_T106E_M148A
(SEQ ID NO: 29)
DKTHTCPPCPAPEAAGGPSVFLFPPKPKDTLMISRTPEVTCVVVDVSHEDPEVKFNWYVDGVEVHNAKTKPREE
QYNSTYRVVSVLTVLHQDWLNGKEYKCQVSNKALPAPIEKTISKAKGQPREPQVYTLPPCRDELTKNQVSLWCL
VKGFYPSDIAVEWESNGQPENNYKTTPPVLDSDGSFFLYSKLTVDKSRWQQGNVFSCSVMHEALHNHYTQKSL
SLSPGKGGSGGSGGSGGSGGSGGSGGSGGSGGSGGSGCDLPQTHSLGSRRTLMLLAQMRKISLFSCLKDRH
DFGFPQEEFGNQFQKAETIPVLHEMIQQIFNLFSTKDSSAAWDETLLDKFYTELYQQLNDLEACVIQGVGVEETP
LMKEDSILAVRKYFQRITLYLKEKKYSPCAWEVVRAEIARSFSLSTNLQESLRSKE
B. hIgG1 Fc_L234A_L235A_K322Q_S354C_T366W-10*GGS-G -hIFNa2_M148A
(SEQ ID NO: 30)
DKTHTCPPCPAPEAAGGPSVFLFPPKPKDTLMISRTPEVTCVVVDVSHEDPEVKFNWYVDGVEVHNAKTKPREE
QYNSTYRVVSVLTVLHQDWLNGKEYKCQVSNKALPAPIEKTISKAKGQPREPQVYTLPPCRDELTKNQVSLWCL
VKGFYPSDIAVEWESNGQPENNYKTTPPVLDSDGSFFLYSKLTVDKSRWQQGNVFSCSVMHEALHNHYTQKSL
SLSPGKGGSGGSGGSGGSGGSGGSGGSGGSGGSGGSGCDLPQTHSLGSRRTLMLLAQMRKISLFSCLKDRH
DFGFPQEEFGNQFQKAETIPVLHEMIQQIFNLFSTKDSSAAWDETLLDKFYTELYQQLNDLEACVIQGVGVTETPL
MKEDSILAVRKYFQRITLYLKEKKYSPCAWEVVRAEIARSFSLSTNLQESLRSKE
C. hIgG1 Fc_L234A_L235A_K322Q_S354C_T366W-10*GGS-G -hIFNa2_T106E_A145G
(SEQ ID NO: 31)
DKTHTCPPCPAPEAAGGPSVFLFPPKPKDTLMISRTPEVTCVVVDVSHEDPEVKFNWYVDGVEVHNAKTKPREE
QYNSTYRVVSVLTVLHQDWLNGKEYKCQVSNKALPAPIEKTISKAKGQPREPQVYTLPPCRDELTKNQVSLWCL
VKGFYPSDIAVEWESNGQPENNYKTTPPVLDSDGSFFLYSKLTVDKSRWQQGNVFSCSVMHEALHNHYTQKSL
SLSPGKGGSGGSGGSGGSGGSGGSGGSGGSGGSGGSGCDLPQTHSLGSRRTLMLLAQMRKISLFSCLKDRH
DFGFPQEEFGNQFQKAETIPVLHEMIQQIFNLFSTKDSSAAWDETLLDKFYTELYQQLNDLEACVIQGVGVEETP
LMKEDSILAVRKYFQRITLYLKEKKYSPCAWEVVRGEIMRSFSLSTNLQESLRSKE
D. hIgG1 Fc_L234A_L235A_K322Q_S354C_T366W-10*GGS-G -hIFNa2_A145G
(SEQ ID NO: 32)
DKTHTCPPCPAPEAAGGPSVFLFPPKPKDTLMISRTPEVTCVVVDVSHEDPEVKFNWYVDGVEVHNAKTKPREE
QYNSTYRVVSVLTVLHQDWLNGKEYKCQVSNKALPAPIEKTISKAKGQPREPQVYTLPPCRDELTKNQVSLWCL
VKGFYPSDIAVEWESNGQPENNYKTTPPVLDSDGSFFLYSKLTVDKSRWQQGNVFSCSVMHEALHNHYTQKSL
SLSPGKGGSGGSGGSGGSGGSGGSGGSGGSGGSGGSGCDLPQTHSLGSRRTLMLLAQMRKISLFSCLKDRH
DFGFPQEEFGNQFQKAETIPVLHEMIQQIFNLFSTKDSSAAWDETLLDKFYTELYQQLNDLEACVIQGVGVTETPL
MKEDSILAVRKYFQRITLYLKEKKYSPCAWEWRGEIMRSFSLSTNLQESLRSKE
E. hIgG1 Fc_L234A_L235A_K322Q_T366Y-10*GGS-G -hIFNa2_T106E_M148A
(SEQ ID NO: 33)
DKTHTCPPCPAPEAAGGPSVFLFPPKPKDTLMISRTPEVTCVVVDVSHEDPEVKFNWYVDGVEVHNAKTKPREE
QYNSTYRVVSVLTVLHQDWLNGKEYKCQVSNKALPAPIEKTISKAKGQPREPQVYTLPPSRDELTKNQVSLYCL
VKGFYPSDIAVEWESNGQPENNYKTTPPVLDSDGSFFLYSKLTVDKSRWQQGNVFSCSVMHEALHNHYTQKSL
SLSPGKGGSGGSGGSGGSGGSGGSGGSGGSGGSGGSGCDLPQTHSLGSRRTLMLLAQMRKISLFSCLKDRH
DFGFPQEEFGNQFQKAETIPVLHEMIQQIFNLFSTKDSSAAWDETLLDKFYTELYQQLNDLEACVIQGVGVEETP
LMKEDSILAVRKYFQRITLYLKEKKYSPCAWEVVRAEIARSFSLSTNLQESLRSKE
F. hIgG1 Fc_L234A_L235A_K322Q_T366Y-10*GGS-G -hIFNa2_M148A
(SEQ ID NO: 34)
DKTHTCPPCPAPEAAGGPSVFLFPPKPKDTLMISRTPEVTCVVVDVSHEDPEVKFNWYVDGVEVHNAKTKPREE
QYNSTYRVVSVLTVLHQDWLNGKEYKCQVSNKALPAPIEKTISKAKGQPREPQVYTLPPSRDELTKNQVSLYCL
VKGFYPSDIAVEWESNGQPENNYKTTPPVLDSDGSFFLYSKLTVDKSRWQQGNVFSCSVMHEALHNHYTQKSL
SLSPGKGGSGGSGGSGGSGGSGGSGGSGGSGGSGGSGCDLPQTHSLGSRRTLMLLAQMRKISLFSCLKDRH
DFGFPQEEFGNQFQKAETIPVLHEMIQQIFNLFSTKDSSAAWDETLLDKFYTELYQQLNDLEACVIQGVGVTETPL
MKEDSILAVRKYFQRITLYLKEKKYSPCAWEVVRAEIARSFSLSTNLQESLRSKE
G. hIgG1 Fc_L234A_L235A_K322Q_T366Y-10*GGS-G -hIFNa2_T106E_A145G
(SEQ ID NO: 35)
DKTHTCPPCPAPEAAGGPSVFLFPPKPKDTLMISRTPEVTCVVVDVSHEDPEVKFNWYVDGVEVHNAKTKPREE
QYNSTYRVVSVLTVLHQDWLNGKEYKCQVSNKALPAPIEKTISKAKGQPREPQVYTLPPSRDELTKNQVSLYCL
VKGFYPSDIAVEWESNGQPENNYKTTPPVLDSDGSFFLYSKLTVDKSRWQQGNVFSCSVMHEALHNHYTQKSL
SLSPGKGGSGGSGGSGGSGGSGGSGGSGGSGGSGGSGCDLPQTHSLGSRRTLMLLAQMRKISLFSCLKDRH
DFGFPQEEFGNQFQKAETIPVLHEMIQQIFNLFSTKDSSAAWDETLLDKFYTELYQQLNDLEACVIQGVGVEETP
LMKEDSILAVRKYFQRITLYLKEKKYSPCAWEVVRGEIMRSFSLSTNLQESLRSKE
H. hIgG1 Fc_L234A_L235A_K322Q_T366Y-10*GGS-G-hIFNa2_A145G
(SEQ ID NO: 36)
DKTHTCPPCPAPEAAGGPSVFLFPPKPKDTLMISRTPEVTCWWVDVSHEDPEVKFNWYVDGVEVHNAKTKPREE
QYNSTYRVVSVLTVLHQDWLNGKEYKCQVSNKALPAPIEKTISKAKGQPREPQVYTLPPSRDELTKNQVSLYCL
VKGFYPSDIAVEWESNGQPENNYKTTPPVLDSDGSFFLYSKLTVDKSRWQQGNVFSCSVMHEALHNHYTQKSL
SLSPGKGGSGGSGGSGGSGGSGGSGGSGGSGGSGGSGCDLPQTHSLGSRRTLMLLAQMRKISLFSCLKDRH
DFGFPQEEFGNQFQKAETIPVLHEMIQQIFNLFSTKDSSAAWDETLLDKFYTELYQQLNDLEACVIQGVGVTETPL
MKEDSILAVRKYFQRITLYLKEKKYSPCAWEVVRGEIMRSFSLSTNLQESLRSKE

To generate human CLEC9A targeted AFNs, any of the above constructs A-D was combined with the CLEC9A VHH-Fc fusion of SEQ ID 2 or 4 resulting in 8 novel constructs. In addition, any of the above constructs E-H was combined with the CLEC9A VHH-Fc fusion of SEQ ID 1 or 3 resulting in an additional set of 8 novel constructs. Proteins were expressed and purified as described in Example 5.

### Example 8: A145G mutation with or without O-glycosylation on T106

In this experiment, Clec9A targeted AFNs with and without T106 O-glycosylation in IFNα2 (R1CHCL50-Fc3+Fc4-IFNα2_A145G versus R1CHCL50-Fc3+Fc4-IFNα2_T106E_A145G), and an untargeted variant (Fc3+Fc4-IFNα2_A145G) were compared. Proteins were produced as described in Example 5 and purified by protein A chromatography followed by size exclusion.

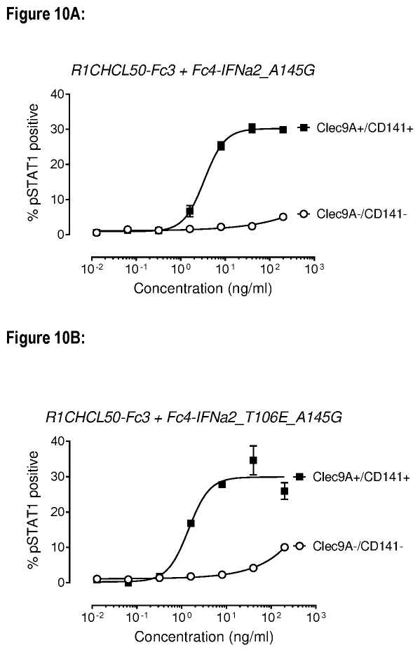

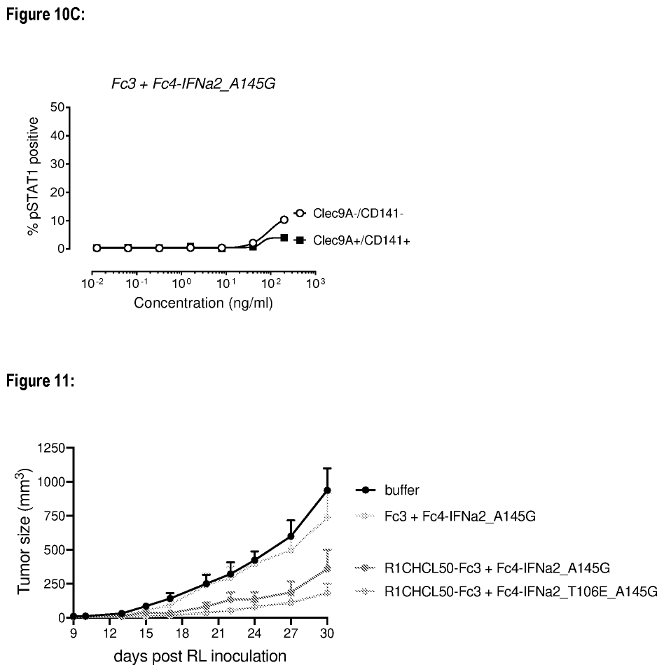

To evaluate the potency the constructs were tested for STAT1 phosphorylation in primary cDC1 (CLEC9A+/CD141+) and non-cDC1 (CLEC9A−/CD141−) populations in human PBMC as described in Example 5. FIG. 10 shows the specificity of the CLEC9A targeted construct with or without O-glycosylation on T106 as this construct is much more potent in activating IFN signaling in cDC1 cells compared to non-cDC1 cells and is much more potent on cDC1 cells compared to cDC1 cells treated with the untargeted variant.

To evaluate the in vivo efficacy of the aforementioned heterodimeric, ‘knob-in-hole’ Fc AFN construct, they were tested in a tumor model in a humanized mouse. In brief, new-born NSG mice (1-2 days of age) were sublethal irradiated with 100 cGy prior to intrahepatic delivery of 1×10⁵ CD34+ human stem cells (from HLA-A2 positive cord bloods). At week 13 after stem cell transfer mice were subcutaneously inoculated with 25×10⁵ human RL follicular lymphoma cells (ATCC CRL-2261; not sensitive to the direct anti-proliferative effect of IFN). Mice were treated intraperitoneally with 30 μg of human Flt3L protein, from day 7 to day 17 after tumor inoculation. Weekly intravenous injection with buffer or Fc-AFN (2.5 μg) constructs was initiated at day 9 after tumor inoculation, when a palpable tumor was visible (n=5 mice per group). Tumor size (caliper measurements), body weight and temperature were assessed daily. Data in FIG. 11 show the tumor growth until one week after the third treatment and demonstrates that both targeted AFNs induced a strong level of tumor growth, while no significant effect was observed with the untargeted variant. Data on body weight and temperature did not show any major difference between buffer treatment and AFN treatment supporting that all AFN treatments were well tolerated.

Sequences
1. P-1479: R1CHCL50-Fc3
(SEQ ID NO: 2)
DVQLVESGGGLVQPGGSLRLSCAASGSFSSINVMGWYRQAPGKERELVARITNLGLPNYADSVKGRFTISRDN
SKNTVYLQMNSLRPEDTAVYYCYLVALKAEYWGQGTLVTVSSGGSGGSGGSGGSGGSDKTHTCPPCPAPEA
AGGPSVFLFPPKPKDTLMISRTPEVTCVVVDVSHEDPEVKFNWYVDGVEVHNAKTKPREEQYNSTYRVVSVLT
VLHQDWLNGKEYKCQVSNKALPAPIEKTISKAKGQPREPQVCTLPPSRDELTKNQVSLSCAVKGFYPSDIAVEW
ESNGQPENNYKTTPPVLDSDGSFFLVSKLTVDKSRWQQGNVFSCSVMHEALHNHYTQKSLSLSPGK
2. P-1542: Fc3
(SEQ ID NO: 38)
DKTHTCPPCPAPEAAGGPSVFLFPPKPKDTLMISRTPEVTCVVVDVSHEDPEVKFNWYVDGVEVHNAKTKPRE
EQYNSTYRVVSVLTVLHQDWLNGKEYKCQVSNKALPAPIEKTISKAKGQPREPQVCTLPPSRDELTKNQVSLSC
AVKGFYPSDIAVEWESNGQPENNYKTTPPVLDSDGSFFLVSKLTVDKSRWQQGNVFSCSVMHEALHNHYTQK
SLSLSPGK
3. P-2157: Fc4-IFNa2_145G
(SEQ ID NO: 32)
DKTHTCPPCPAPEAAGGPSVFLFPPKPKDTLMISRTPEVTCVVVDVSHEDPEVKFNWYVDGVEVHNAKTKPREEQYNSTY
RVVSVLTVLHQDWLNGKEYKCQVSNKALPAPIEKTISKAKGQPREPQVYTLPPCRDELTKNQVSLWCLVKGFYPSDIAVEW
ESNGQPENNYKTTPPVLDSDGSFFLYSKLTVDKSRWQQGNVFSCSVMHEALHNHYTQKSLSLSPGKGGSGGSGGSGGS
GGSGGSGGSGGSGGSGGSGCDLPQTHSLGSRRTLMLLAQMRRISLFSCLKDRHDFGFPQEEFGNQFQKAETIPVLHEMI
QQIFNLFSTKDSSAAWDETLLDKFYTELYQQLNDLEACVIQGVGVTETPLMKEDSILAVRKYFQRITLYLKEKKYSPCAWEW
RGEIMRSFSLSTNLQESLRSKE
4. P-2158: Fc4-IFNa2_T106E_145G
(SEQ ID NO: 31)
DKTHTCPPCPAPEAAGGPSVFLFPPKPKDTLMISRTPEVTCVVVDVSHEDPEVKFNWYVDGVEVHNAKTKPRE
EQYNSTYRVVSVLTVLHQDWLNGKEYKCQVSNKALPAPIEKTISKAKGQPREPQVYTLPPCRDELTKNQVSLW
CLVKGFYPSDIAVEWESNGQPENNYKTTPPVLDSDGSFFLYSKLTVDKSRWQQGNVFSCSVMHEALHNHYTQ
KSLSLSPGKGGSGGSGGSGGSGGSGGSGGSGGSGGSGGSGCDLPQTHSLGSRRTLMLLAQMRRISLFSCLK
DRHDFGFPQEEFGNQFQKAETIPVLHEMIQQIFNLFSTKDSSAAWDETLLDKFYTELYQQLNDLEACVIQGVGV
EETPLMKEDSILAVRKYFQRITLYLKEKKYSPCAWEVVRGEIMRSFSLSTNLQESLRSKE

### Example 9: Fc-AFN Constructs

Mass spectrometry analysis illustrated that the C-terminal lysine K residue in the R1CHCL50-Fc3 chain is cleaved off in almost all mature proteins. Therefore, variants are constructed in which this lysine residue in both Fc-chains was removed. Resulting proteins will be referred to as Fc′ proteins. By way of example, the sequences for the chimeric protein combination of R1CHCL50-Fc3′ with Fc4′-AFN fusions in which residue A145 was mutated to G in IFNa2b, or in which the residues T106 and A145 were mutated to respectively E and G in IFNa2a, are shown below.

Sequences:
P-2379: Fc4′-IFNa2b_A145G
(SEQ ID NO: 41)
DKTHTCPPCPAPEAAGGPSVFLFPPKPKDTLMISRTPEVTCWWVDVSHEDPEVKFNWYVDGVEVHNAKTKPREE
QYNSTYRVVSVLTVLHQDWLNGKEYKCQVSNKALPAPIEKTISKAKGQPREPQVYTLPPCRDELTKNQVSLWCL
VKGFYPSDIAVEWESNGQPENNYKTTPPVLDSDGSFFLYSKLTVDKSRWQQGNVFSCSVMHEALHNHYTQKSL
SLSPGGGSGGSGGSGGSGGSGGSGGSGGSGGSGGSGCDLPQTHSLGSRRTLMLLAQMRRISLFSCLKDRHD
FGFPQEEFGNQFQKAETIPVLHEMIQQIFNLFSTKDSSAAWDETLLDKFYTELYQQLNDLEACVIQGVGVTETPL
MKEDSILAVRKYFQRITLYLKEKKYSPCAWEVVRGEIMRSFSLSTNLQESLRSKE
P-2380: Fc4'-IFNa2a_T106E_A145G
(SEQ ID NO: 42)
DKTHTCPPCPAPEAAGGPSVFLFPPKPKDTLMISRTPEVTCVVVDVSHEDPEVKFNWYVDGVEVHNAKTKPREE
QYNSTYRVVSVLTVLHQDWLNGKEYKCQVSNKALPAPIEKTISKAKGQPREPQVYTLPPCRDELTKNQVSLWCL
VKGFYPSDIAVEWESNGQPENNYKTTPPVLDSDGSFFLYSKLTVDKSRWQQGNVFSCSVMHEALHNHYTQKSL
SLSPGGGSGGSGGSGGSGGSGGSGGSGGSGGSGGSGCDLPQTHSLGSRRTLMLLAQMRKISLFSCLKDRHD
FGFPQEEFGNQFQKAETIPVLHEMIQQIFNLFSTKDSSAAWDETLLDKFYTELYQQLNDLEACVIQGVGVEETPL
MKEDSILAVRKYFQRITLYLKEKKYSPCAWEVVRGEIMRSFSLSTNLQESLRSKE
P-1479b: R1CHCL50-Fc3″
(SEQ ID NO: 43)
DVQLVESGGGLVQPGGSLRLSCAASGSFSSINVMGWYRQAPGKERELVARITNLGLPNYADSVKGRFTISRDNS
KNTVYLQMNSLRPEDTAVYYCYLVALKAEYWGQGTLVTVSSGGSGGSGGSGGSGGSDKTHTCPPCPAPEAAG
GPSVFLFPPKPKDTLMISRTPEVTCVVVDVSHEDPEVKFNWYVDGVEVHNAKTKPREEQYNSTYRVVSVLTVLH
QDWLNGKEYKCQVSNKALPAPIEKTISKAKGQPREPQVCTLPPSRDELTKNQVSLSCAVKGFYPSDIAVEWESN
GQPENNYKTTPPVLDSDGSFFLVSKLTVDKSRWQQGNVFSCSVMHEALHNHYTQKSLSLSPG

What is claimed is:

1.-48. (canceled)

49. A chimeric protein complex comprising:
(i) a targeting moiety that specifically binds to C-type lectin domain family 9 member A (Clec9A) comprising a recombinant heavy-chain-only antibody (VHH) that has an amino acid sequence of at least 95% identity with SEQ ID NO: 11 or 12,
(ii) a mutated human IFNα2 having an amino acid sequence having at least 95% identity with SEQ ID NO: 9 or 10, and
(iii) a mutated Fc domain comprising a mutation selected from P329G, K322Q, K322A, and P331S relative to any one of SEQ ID NOs: 13-16,
wherein the chimeric protein complex comprises a polypeptide having an amino acid sequence having at least 95% identity with any one of SEQ ID NOs: 2, 1, 3, 4, and 43 and further comprises a polypeptide having an amino acid sequence having at least 95% identity with any one of SEQ ID NOs: 7, 5, 6, 8, 29-36, 41 and 42.

50. The chimeric protein complex of claim 49, wherein the chimeric protein complex is a heterodimer.

51. The chimeric protein complex of claim 49, wherein the mutated human IFNα2 has an amino acid sequence having at least 98% identity with SEQ ID NO: 9 or 10.

52. The chimeric protein complex of claim 51, wherein the mutated human IFNα2 has an amino acid sequence having at least 99% identity with SEQ ID NO: 9 or 10.

53. The chimeric protein complex of claim 49, wherein the mutated human IFNα2 has 1-3 mutations relative to the amino acid sequence of SEQ ID NO: 9 or 10.

54. The chimeric protein complex of claim 53, wherein the mutated human IFNα2 has a R149A mutation with respect to SEQ ID NO: 9 or 10 or one of a A145G, R33A, R144A, R144I, R144L, R144S, R144T, R144Y, A145D, A145H, A145K, A145Y, M148A and L153A mutation with respect to SEQ ID NO: 9 or 10.

55. The chimeric protein complex of claim 49, wherein the VHH has an amino acid sequence of at least 98% identity with SEQ ID NO: 11 or 12.

56. The chimeric protein complex of claim 55, wherein the VHH has an amino acid sequence of at least 99% identity with SEQ ID NO: 11 or 12.

57. The chimeric protein complex of claim 56, wherein the VHH has the amino acid sequence of SEQ ID NO: 11 or 12.

58. The chimeric protein complex of claim 57, wherein the modified Fc domain has one or more of the following mutations: P329G, K322Q, K322A, or P331S relative to human IgG1 Fc.

59. The chimeric protein complex of claim 49, wherein the modified Fc domain has an amino acid sequence of at least 93% identity with SEQ ID NO: 15, 16, 13, or 14.

60. The chimeric protein complex of claim 59, wherein the modified Fc domain has an amino acid sequence of at least 95% identity with SEQ ID NO: 15, 16, 13, or 14.

61. The chimeric protein complex of claim 60, wherein the modified Fc domain has an amino acid sequence of at least 98% identity with SEQ ID NO: 15, 16, 13, or 14.

62. A pharmaceutical composition comprising the chimeric protein complex of claim 49 and a pharmaceutically acceptable carrier.

63. A method for treating or preventing cancer, the method comprising administering the pharmaceutical composition of claim 62 to a patient in need thereof.

64. The method of claim 63, wherein the cancer is selected from one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer;
uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

65. A recombinant nucleic acid composition encoding one or more chimeric protein complexes of claim 49, or constituents thereof.

66. A host cell comprising the recombinant nucleic acid composition of claim 65.


SEQUENCE LISTING (43 sequences)

SEQ ID NO: 1 - protein, 357 aa, synthetic construct
  REGION 1..357: note = Synthetic Sequence
DVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 2 - protein, 357 aa, synthetic construct
  REGION 1..357: note = Synthetic Sequence
DVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3 - protein, 357 aa, synthetic construct
  REGION 1..357: note = Synthetic Sequence
DVQLVESGGGLVQPGGSLRLSCAASGRIFSVNAMGWYRQAPGKQRELVAAITNQGAPTYADSVKGRFTISRDNSKNTVYLQMNSLRPEDTAVYYCKAFTRGDDYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 4 - protein, 357 aa, synthetic construct
  REGION 1..357: note = Synthetic Sequence
DVQLVESGGGLVQPGGSLRLSCAASGRIFSVNAMGWYRQAPGKQRELVAAITNQGAPTYADSVKGRFTISRDNSKNTVYLQMNSLRPEDTAVYYCKAFTRGDDYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 5 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 6 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 7 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 8 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 9 - protein, 165 aa, synthetic construct
  REGION 1..165: note = Synthetic Sequence
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 10 - protein, 165 aa, synthetic construct [sequence omitted]

SEQ ID NO: 11 - protein, 115 aa, synthetic construct
  REGION 1..115: note = Synthetic Sequence
DVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSS

SEQ ID NO: 12 - protein, 115 aa, synthetic construct
  REGION 1..115: note = Synthetic Sequence
DVQLVESGGGLVQPGGSLRLSCAASGRIFSVNAMGWYRQAPGKQRELVAAITNQGAPTYADSVKGRFTISRDNSKNTVYLQMNSLRPEDTAVYYCKAFTRGDDYWGQGTLVTVSS

SEQ ID NO: 13 - protein, 227 aa, synthetic construct
  REGION 1..227: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 14 - protein, 227 aa, synthetic construct
  REGION 1..227: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 227 aa, synthetic construct
  REGION 1..227: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 16 - protein, 227 aa, synthetic construct
  REGION 1..227: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 487 aa, synthetic construct
  REGION 1..487: note = Synthetic Sequence
DVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 18 - protein, 487 aa, synthetic construct
  REGION 1..487: note = Synthetic Sequence
DVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGSGGSGGSGGSGGSDVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSS

SEQ ID NO: 19 - protein, 553 aa, synthetic construct
  REGION 1..553: note = Synthetic Sequence
DVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 20 - protein, 487 aa, synthetic construct
  REGION 1..487: note = Synthetic Sequence
QVQLQESGGGLVQAGGSLRLSCTASGTIFSINRMDWFRQAPGKQRELVALITSDGTPAYADSAKGRFTISRDNTKKTVSLQMNSLKPEDTAVYYCHVSSGVYNYWGQGTQVTVSSGGSGGSGGSGGSGGSDVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 487 aa, synthetic construct
  REGION 1..487: note = Synthetic Sequence
DVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGSGGSGGSGGSGGSQVQLQESGGGLVQAGGSLRLSCTASGTIFSINRMDWFRQAPGKQRELVALITSDGTPAYADSAKGRFTISRDNTKKTVSLQMNSLKPEDTAVYYCHVSSGVYNYWGQGTQVTVSS

SEQ ID NO: 22 - protein, 553 aa, synthetic construct
  REGION 1..553: note = Synthetic Sequence
QVQLQESGGGLVQAGGSLRLSCTASGTIFSINRMDWFRQAPGKQRELVALITSDGTPAYADSAKGRFTISRDNTKKTVSLQMNSLKPEDTAVYYCHVSSGVYNYWGQGTQVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 23 - protein, 647 aa, synthetic construct
  REGION 1..647: note = Synthetic Sequence
MKLPVRLLVLMFWIPASSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSQVQLQESGGGLVQPGGSLRLSCAASGRIFSVNAMGWYRQAPGKQRELVAAITNQGAPTYADSVKGRFTISRDNAGNTVYLQMNSLRPEDTAVYYCKAFTRGDDYWGQGTQVTVSSGGSGGSGGSGGSGGSGGSGGSGGSGGSGGSGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 24 - protein, 647 aa, synthetic construct
  REGION 1..647: note = Synthetic Sequence
MGWSCIIFFLVATATGVHSQVQLQESGGGLVQPGGSLRLSCAASGRIFSVNAMGWYRQAPGKQRELVAAITNQGAPTYADSVKGRFTISRDNAGNTVYLQMNSLRPEDTAVYYCKAFTRGDDYWGQGTQVTVSSGGSGGSGGSGGSGGSGGSGGSGGSGGSGGSGGSGGSGGSGGSGGSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 25 - protein, 353 aa, synthetic construct
  REGION 1..353: note = Synthetic Sequence
QVQLQESGGGLVQPGGSLRLSCAASGRIFSVNAMGWYRQAPGKQRELVAAITNQGAPTYADSVKGRFTISRDNAGNTVYLQMNSLRPEDTAVYYCKAFTRGDDYWGQGTQVTVSSVDGGSGGSGGSGGSGGSGGSRSGGSGGSGGSGGSGGSGGSGGSGGSGGSGGSGGSGGSGGSAAAMCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKELEHHHHHH

SEQ ID NO: 26 - protein, 402 aa, synthetic construct
  REGION 1..402: note = Synthetic Sequence
QVQLQESGGGLVHPGGSLRLSCAASGSFSSINVMGWYRQAPGKERELVARITNLGLPNYADSVTGRFTISRDNAKNTVYLQMNSLKPEDTAVYYCYLVALKAEYWGQGTQVTVSSGGSGGSGGSGGSGGSGGSGGSGGSGGSGGSGGSGGSGGSGGSGGSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 27 - protein, 452 aa, synthetic construct
  REGION 1..452: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRGEIMRSFSLSTNLQESLRSKE

SEQ ID NO: 28 - protein, 452 aa, synthetic construct
  REGION 1..452: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIARSFSLSTNLQESLRSKE

SEQ ID NO: 29 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIARSFSLSTNLQESLRSKE

SEQ ID NO: 30 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIARSFSLSTNLQESLRSKE

SEQ ID NO: 31 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRGEIMRSFSLSTNLQESLRSKE

SEQ ID NO: 32 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRGEIMRSFSLSTNLQESLRSKE

SEQ ID NO: 33 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIARSFSLSTNLQESLRSKE

SEQ ID NO: 34 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIARSFSLSTNLQESLRSKE

SEQ ID NO: 35 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRGEIMRSFSLSTNLQESLRSKE

SEQ ID NO: 36 - protein, 423 aa, synthetic construct
  REGION 1..423: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRGEIMRSFSLSTNLQESLRSKE

SEQ ID NO: 37 - skipped

SEQ ID NO: 38 - protein, 227 aa, synthetic construct
  REGION 1..227: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 39-40 - skipped (2 entries)

SEQ ID NO: 41 - protein, 422 aa, synthetic construct
  REGION 1..422: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGSGGSGGSGGSGGSGGSGGSGGSGGSGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRGEIMRSFSLSTNLQESLRSKE

SEQ ID NO: 42 - protein, 422 aa, synthetic construct
  REGION 1..422: note = Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGSGGSGGSGGSGGSGGSGGSGGSGGSGCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRGEIMRSFSLSTNLQESLRSKE

SEQ ID NO: 43 - protein, 356 aa, synthetic construct
  REGION 1..356: note = Synthetic Sequence
DVQLVESGGGLVQPGGSLRLSCAASGSFSSINVMGWYRQAPGKERELVARITNLGLPNYADSVKGRFTISRDNSKNTVYLQMNSLRPEDTAVYYCYLVALKAEYWGQGTLVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG